(19) United States
(12) Patent Application Publication
(10) Pub. No.: US 2021/0187039 A1
(43) Pub. Date: Jun. 24, 2021

(54) FECAL PROCESSING DEVICE AND METHODS

(71) Applicants: The Medical Research, Infrastructure and Health Services Fund of the Tel Aviv Medical Center, Tel-Aviv (IL); Fund for Medical Research Development of Infrastructure & Health Services by Barzilai Medical Center, Ashkelon (IL)

(72) Inventors: Arie FELDMAN, Ashkelon (IL); Nitsan MAHARSHAK, Herzliya (IL); David GALES, Ashkelon (IL)

(73) Assignees: The Medical Research, Infrastructure and Health Services Fund of the Tel Aviv Medical Center, Tel-Aviv (IL); Fund for Medical Research Development of Infrastructure & Health Services by Barzilai Medical Center, Ashkelon (IL)

(21) Appl. No.: 17/269,289

(22) PCT Filed: Aug. 20, 2019
(86) PCT No.: PCT/IL2019/050931

§ 371 Date: Feb. 18, 2021

Related U.S. Application Data
(60) Provisional application No. 62/719,794, filed on Aug. 20, 2018.

(51) Int. Cl. A61K 35/37; B01L 3/00; B01D 29/90; B01D 29/085

(52) U.S. Cl. CPC A61K 35/37; B01L 3/50853; B01L 3/5635; A61B 10/0038; B01D 29/085; B01L 2300/0681; B01D 29/902

(57) ABSTRACT

A method of producing an implant for fecal transplant (FT), including: introducing fecal materials into a container, and processing the fecal materials to a smaller size in the container. According to some embodiments, the container is in ambient conditions, and the processing in the container includes non-ambient conditions. Optionally, the non-ambient conditions include an atmosphere having at most 18% of O₂. Optionally, the method includes adding a liquid into the container, the liquid including a preservative material. Optionally, the liquid includes between 10% to 30% glycerol by volume. Optionally, the method includes sealing the container with a sealing lid after the introducing, and processing the fecal materials in the sealed container. Optionally, the processing of the fecal materials includes cutting the fecal materials by a cutting element to increase an exposed surface area of the fecal materials.

### RELATED APPLICATION

This application claims the benefit of priority of U.S. Provisional Patent Application No. 62/719,794 filed on 20 Aug. 2018, the contents of which are incorporated herein by reference in their entirety.

### FIELD AND BACKGROUND

The present invention, in some embodiments thereof, relates to fecal materials and microbiota transplantation and more particularly, but not exclusively, to a device and method for preparing fecal materials for a fecal transplant (FT).

Background art includes U.S. Patent No. US20140238154 describes “a fecal microbiome transplant material preparation method and apparatus for the preparation of fecal material for a fecal transplant. A healthy patient's stool is collected and deposited in a first container which has its open end attached to a second container to form a fecal processing device. Blender blades are positioned in the second container and a filter is mounted therein. A liquid is injected into the second container through a syringe port having a valve therein to block the escape of liquid from the connected containers. Intake and exhaust ports allow air to enter and escape from the connected containers while blocking the escape of liquid. The liquid and fecal matter in the connected containers are blended and the blended liquid is strained through the filter in the second container and removed through the syringe port for use in a fecal transplant”.

U.S. Pat. No. 4,170,798 describes an “apparatus to receive fecal material directly into a container and maintain it isolated, particularly as to its odors, while automatically homogenizing and withdrawing samples as may be desired and disposing of the remainder. The apparatus, either in fixed or portable form, includes connections to a source of homogenization fluid, a source of cleansing water, and a drain. It also includes means to mix the fecal material vigorously with the homogenization fluid to achieve a substantially homogeneous mixture which can be disposed of with relative ease and which can be sampled into a vacutainer through a vacutainer needle attached liquid-tight to the chamber containing the mixture. For multiple sampling, an automatically operated vacutainer changer brings one vacutainer at a time into position to have a sample of the mixture injected into it. Following such sampling, the apparatus and needle are automatically washed”.

U.S. Pat. No. 9,486,183 describes an “apparatus for the collection of donated stool, a collection chamber for depositing (or collecting), processing and harvesting stool from a donor and a process for the preparation of a composition comprising donated stool and a pharmaceutically acceptable carrier”.

Patent Application Publication No. WO2017051413 describes a “system, means and methods for collecting, sampling and testing stool samples. The publication pertains to both mechanical and electrical, manual, semi-automatic, automatic means and methods for automatic sampling and homogenization or extraction of the stool samples, enabling their usability in various diagnosis and detection applications”.

U.S. Patent Application Publication No. US 20160206293 describes a “technology relating to collecting and containing samples and particularly, but not exclusively, to technology for collecting and containing a stool specimen. The technology provides a device for collecting and containing a stool specimen, the device comprising ergonomic features optimized for a geriatric user. The technology also provides an enclosing holder for securing the ergonomic stool specimen container in a leak-proof manner e.g., during mechanical shaking”.

Chinese Patent No. CN201765132 describes a device for collecting and preprocessing feces which comprises a plastic sampling rod component and a sample processing tube component.

U.S. Pat. No. 8,491,498 describes “A sample collection implement S1 includes a container 1 having an accommodation portion 10 in which a liquid 5 for suspending or diluting a sample is accommodated, a sample collection stick 2 being able to be disposed in the accommodation portion 10, a filter 3 provided inside the container 1, and a movable member 4 that can be moved in a predetermined direction inside the container land has a function of pushing the liquid 5 and causing the liquid to pass through the filter 3, when moved in the predetermined direction. With such a configuration, when the liquid 5 is filtered, it is not necessary to move the filter 3 by directly pushing it and a filter with a low mechanical strength can be used as the filter 3. In addition, it is not necessary to use a centrifugal apparatus.”

U.S. Pat. No. 8,597,954 describes “The present invention relates to a stool sample processing method and stool sample processing container provided with a stool collection tool, a suspending solution holding portion and a processing solution holding portion, wherein stool sample preparation solutions consisting of a suspending solution and a stool sample processing solution are respectively housed in a suspending solution holding container and a processing solution holding container, after first mixing a stool sample with the suspending solution and suspending therein, a sealant is released into the suspending solution holding container by pressing on the lower portion of the processing solution holding container, and the resulting stool suspension mixes with the stool sample processing solution that stabilizes the nuclide acid”.

JP Patent No. JP2009115658 describes a “method for the sample feces, the collected sample feces are formed into an easily crushable shape such as a plate-like shape having 0.5-5 mm of thickness, a fibrous shape having 0.5-5 mm of diameter and a granular shape having 1-10 mm of diameter, to be frozen thereafter. This preparation container for the sample feces has a cylindrical member 11 deformable elastically and provided with a plurality of recesses 11a1 for collecting feces, on a side face, a removing means 12 for removing excessive feces E out of the feces collected in the recesses 11a1 of the cylindrical member 11, and an extrusion member 13 capable of press-deforming the recesses 11a1 by insertion thereof from an inner side of the cylindrical member 11.”

Scandinav J. Clin. 6. Lab. Investigation 416-417, 14, 1962, by Isaksson et al describes a “simple device for homogenizing and sampling of feces in a closed system.

The increasing clinical demand for quantitative analyses of feces makes it desirable to make the rather unpleasant homogenizing procedure less annoying. The device to be described, designed in our laboratory, is an attempt in that direction. It permits the procedure to be carried out in a collection vessel which is part of a closed system. The samples to be analyzed are taken with minimal air-contact. The same is true for the cleaning procedure.”

### SUMMARY

The following describe some examples of embodiments of the invention. Other embodiments are within the scope of the description, including embodiments in which only some of the features from one example are used. Other embodiments are within the scope of the description, including examples in which some of the features are selected from two or more examples.

Example 1. A method of producing an implant for fecal transplant (FT), comprising: introducing fecal materials into a container, and processing said fecal materials to a smaller size in said container. According to some embodiments, the container is in ambient conditions, and said processing in said container comprises non-ambient conditions.

Example 2. The method of example 1, wherein said non-ambient conditions comprising an atmosphere having at most 18% of O₂.

Example 3. The method of examples 1-2, comprising adding a liquid into said container, said liquid comprising a preservative material.

Example 4. The method of example 3, wherein said liquid comprises between 10% to 30% glycerol by volume.

Example 5. The method of examples 1-4, further comprising sealing said container with a sealing lid after said introducing, and processing said fecal materials in said sealed container.

Example 6. The method of any one of examples 1-5, wherein said processing of said fecal materials comprises cutting said fecal materials by a cutting element to increase an exposed surface area of said fecal materials.

Example 7. The method of any one of examples 1 to 6, wherein said container comprises a first upper compartment and a second lower compartment, and a separating screen separating said compartments.

Example 8. The method of example 7, wherein said processing further comprising moving said separating screen in relation to a blocker, to allow transition of said fecal materials from the upper compartment to the lower compartment.

Example 9. The method of example 8, wherein said method further comprises: a) connecting a handle to a sealing lid; b) after a), rotating said handle to rotate said processing element; and c) after b), rotating said handle to move said separating screen in relation to said blocker.

Example 10. The method of example 9, wherein said method further comprising moving said handle between two adaptors.

Example 11. The method of any one of examples 7-10, comprising conveying said fecal materials through a plurality of apertures located in said separating screen downward to said lower compartment.

Example 12. The method of example 11, wherein said fecal materials are reduced to a size which is smaller than 8 mm.

Example 13. The method of any one of examples 8-12, further comprising directing said fecal materials along an inner perimeter of said blocker, said inner perimeter having a funneled geometry to allow conveyance of said liquid toward a filter, wherein said blocker is positioned below said separating screen.

Example 14. The method of any one of examples 8-13, wherein said blocker has a conical shape.

Example 15. The method of example 13, further comprising conveying said fecal materials through said filter, wherein said filter blocks the transition of elements that have a maximal extent larger than 2 mm.

Example 16. The method of any one of examples 13-15, wherein said filter is positioned below said blocker and above a collecting compartment, further comprising conveying said fecal materials toward said filter to said collecting compartment.

Example 17. The method of example 16, further comprising removing said fecal materials from said collecting compartment through a valve to a storage unit.

Example 18. Apparatus for producing implant for fecal transplant (FT), comprising: a container sized to receive a sample of fecal materials, and a size reducer positioned in said container and operable to reduce the size of said fecal materials. Wherein, said container comprises a separating screen which divides said container into a first upper compartment comprising said size reducer and to a second lower compartment. According to some embodiments, said separating screen is positioned onto a blocker, wherein said separating screen is movable in relation to said blocker to allow transition of liquids and fecal materials between said first upper compartment and said second lower compartment.

Example 19. The apparatus of example 18, further comprising a sealing lid, said sealing lid is sized and shaped to prevent transition of gases and liquids between an inner section of said container and outer environment.

Example 20. The apparatus of examples 18-19, wherein said size reducer comprises at least one rotating cutting blade.

Example 21. The apparatus of examples 18-20, wherein said container further comprises a collecting compartment, said collecting compartment is positioned under said lower compartment and separated from said lower compartment by a filter.

Example 22. The apparatus of examples 18-21, wherein said separating screen comprises a plurality of apertures having a maximal extent of at most 8 mm, said apertures are selectively blocked by said blocker.

Example 23. The apparatus of examples 18 to 22, wherein said container is shaped and sized for insertion into a toilet bowl.

Example 24. The apparatus of example 19, wherein said sealing lid comprising a handle attached to said lid, and wherein said lid comprises: a first top surface, a second bottom surface, transmission positioned between said first top surface and said second bottom surface. According to some embodiments, said transmission transfers a rotation of said handle to said size reducer.

Example 25. The apparatus of example 21, wherein said blocker has an inner perimeter having a conical shape, configured to direct said liquids and fecal materials gravitationally toward said filter.

Example 26. The apparatus of examples 21-25, wherein said filter comprising pores having a maximal extent of at most 2 mm.

Example 27. The apparatus of example 21, further comprising a valve to allow removing of said fecal materials from said colleting compartment.

Example 28. The apparatus of examples 18-27, wherein said separating screen is interconnected to a shaft, said shaft is configured to move said separating screen in relation to said blocker.

Example 29. The apparatus of example 24, wherein said size reducer is interconnected to said transmission, said transmission is configured to rotate said size reducer.

Example 30. Apparatus for producing implant for fecal transplant (FT), having a processing container sized to receive a sample of fecal materials; a processing element, positioned inside the container to reduce the size of the fecal materials; a compartment for receiving processed fecal materials; and one or more outlets at one or more walls of the compartment to extract the fecal materials out of the container. In some embodiments, the one or more outlets have a maximal width of less than 8 mm.

Example 31. A fecal transplantation kit, having a fecal processing apparatus according to Example 30, and one or more storage units, comprising a port sized and shaped to be secured to one or more outlets without a leakage of the fecal material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a process of preparation of fecal materials for a fecal transplant, according to some embodiments of the invention;

FIG. 2 is a block diagram of a device for the preparation of fecal materials for a fecal transplant, according to some embodiments of the invention;

FIG. 3 is a general flowchart of a fecal materials processing and device operation method for the preparation a fecal transplant, according to some embodiments of the invention;

FIG. 4 is a flow chart of a process for the preparation of fecal materials for a fecal transplant, including examples of actions taken by a user, according to some embodiments of the invention;

FIG. 5A is a block diagram of a general fecal processing device including processing fecal materials by rotation of an external handle, according to some embodiments of the invention;

FIG. 5B is a block diagram of a general fecal processing device including moving a separation screen by rotation of an external handle, according to some embodiments of the invention;

FIG. 6A is a perspective side longitudinal view of a fecal processing device in an assembled configuration, according to some embodiments of the invention;

FIG. 6B is a perspective view of a fecal processing device in a disassembled configuration, according to some embodiments of the invention;

FIG. 7A is a perspective view of a sealing lid in an assembled configuration, according to some embodiments of the invention;

FIG. 7B is a perspective view of a sealing lid in a disassembled configuration, according to some embodiments of the invention;

FIG. 8 is a side cross-sectional view of the fecal processing device, according to some embodiments of the invention;

FIG. 9A is a side view illustrations of an exemplary fecal processing device, having a processing container positioned inside a filtering container, according to some embodiments of the invention;

FIGS. 9B-C are side views of an exemplary fecal processing device, having a processing container positioned inside a filtering container in a coupled position (FIG. 9B) and in an un-coupled position (FIG. 9C), according to some embodiments of the invention;

FIG. 10A is a simplified flowchart of a process of storing processed fecal, according to some embodiments of the invention;

FIG. 10B is a flowchart of a method of transplanting processed fecal stored in a fecal material storage unit, according to some embodiments of the invention;

FIGS. 11A to 11E are schematic illustrations of a fecal material storage unit, according to some embodiments of the invention; and

FIG. 12 is a simplified flowchart of a process of storing and transplanting processed fecal, according to some embodiments of the invention.

### DESCRIPTION OF EMBODIMENTS OF THE INVENTION

The present invention, in some embodiments thereof, relates to a fecal materials and microbiota transplantation and more particularly, but not exclusively, to a device and method for preparing fecal materials for a fecal transplant (FT).

### Overview

A broad aspect of some embodiments of the invention relates to preparing a fecal transplant under supportive environmental conditions.

In some embodiments, gaseous and/or liquid and/or temperature and/or toxins and/or supportive materials and/or growth factor and/or nutrients are provided, controlled and/or modified during at least some parts of FT preparation procedure, for example, during collecting, processing, storing, and/or during transplanting fecal transplant to a recipient. These conditions may, for example, assist in maintaining, modifying, and/or encouraging a desired mix of microbes and/or other biological material for the transplant.

An aspect of some embodiments of the invention relates to preparation of a fecal transplant in a non-ambient environment conditions, for example within a reduced-oxygen environmental conditions, during at least some parts of a preparation procedure of FT preparation.

In some embodiments, reduced oxygen environmental conditions are achieved for example by placing fecal materials in a low oxygen atmospheric environment, where the relative concentration of oxygen is lower than its concentration in ambient atmospheric conditions. For example lower than 18%. In another example, oxygen concentration is between 3% to 18%, between 3% to 10%, between 3% to 8%, or any intermediate, smaller or larger value, or range of values of concentrations.

Optionally, the fecal transplant is prepared in conditions wherein the concentration of oxygen is similar to or lower than its concentration in the relevant extracted microorganism intra-body habitat e.g., between 3-18% oxygen.

A potential advantage of reducing exposure to oxygen, is reducing degradation of at least some oxygen-sensitive microorganisms which are collected as part of the fecal materials.

In some embodiments, exposure to oxygen is reduced by collecting fecal materials immediately from a donor, for example, by placing a container in a toilet bowl, which is sized and shaped for fecal materials collection and collect fecal materials from the donor during a natural defecation.

Optionally or alternatively, exposure is reduced by collecting fecal materials directly from a donor's intestine, by extracting fecal materials directly from the donor's intestine.

In some embodiments, exposure to oxygen is reduced by placing fecal materials in a sealed container so as to differentiate between the gas composition in the outer environment and the gas composition which is introduce into the container.

Exposure of fecal materials to a desired gas atmosphere is enhanced for example, by introducing a desired gas to the sealed container, wherein the fecal materials are placed. This gas may mix with and/or its introduction may expel existing gas.

In some embodiments, both microorganisms which are positioned at the surface area of the introduced fecal material and microorganisms which are positioned at the inner sections of the fecal material are exposed to a desired gas atmosphere. This may be achieved, for example, by modifying the geometry of the fecal materials e.g., by reducing the size of the fecal materials to smaller parts.

An aspect of some embodiments of the invention relates to producing a dispersion of fecal materials in a liquid medium.

In some embodiments, the liquid medium is introduced prior and/or during the FT preparation process, for example, during collecting, processing, storage, and/or during transplanting the fecal materials to a recipient.

A potential advantage of dispersing fecal materials in a liquid medium is obtaining a homogeneous solution having dispersed microorganisms therein.

This homogeneous solution may be divided into smaller portions having relatively similar content of microorganism therein, which may be administered by the recipient to obtain an effective therapeutic regimen. For example, a 25 ml sample of fecal transplant a day, during a period of six months. In some embodiments, a preferred therapeutic protocol depends on the indication. For example, patients who suffer from a resistant “Clostridium difficile” infection, a single FMT (fecal microbial transplantation) will be beneficial in up to 70% of the patients.

In some embodiments, the portions may be stored in storage units, for example in a container and/or in capsules and/or in an enema bag.

In some embodiments, fecal materials are placed in a supportive liquid medium which contains preservatives to prevent degradation of at least some of the microorganisms that may be sensitive to a dry environmental conditions and/or to a prolonged storage period. In some embodiments, such medium may contain glycerol and/or saline and/or water.

In some embodiments, the temperature of the surrounding medium (either a liquid and/or gaseous surrounding) is modified. A potential advantage of modifying the temperature is reduction degradation of at least some sensitive microorganisms within the fecal materials which may be sensitive to certain range of temperatures.

Optionally, the temperature is modified to a temperature which is below an ambient temperature. Optionally or alternatively, the temperature is modified to a temperature which is below 0° C.

In some embodiments, fecal transplants can be stored in cold environment conditions for a prolonged storage period. For example, fecal transplants can be safely stored at −20° C. for 6 months. In another example, fecal transplants can be safely stored at −80° C. for 2 years.

According to some embodiments, storing processed fecal material is by coupling a storage unit to a fecal processing device, extracting the processes fecal into the storage unit, and disposing the storage unit in low temperature environment. In some embodiments, storing is performed by the patient.

According to some embodiments, the storage unit has a port shaped to fit an outlet at the fecal processing device without leakage. In some embodiments, the system provides an indication to the user, when coupling is proper and complete. In some embodiments, the fecal processing device includes an outlet valve to control flow of processed fecal towards the storage unit. In some embodiments, the fecal processing device includes a pressure valve to balance pressure within the fecal processing device. According to some embodiments, coupling is without leaking processed fecal outside the processing device and the storage unit. In some embodiments, coupling is by screwing the port at the storage unit to the outlet at the processing device. In some embodiments, coupling is by pressing a port at the storage unit into/over an outlet at the processing device. In some embodiments, coupling is by an adaptor interconnecting the processing device and the storage unit.

According to some embodiments, the storage unit includes a manual pump for extracting processes fecal outside the fecal processing. In some embodiments, the storage unit includes a manual pump to force/inject the stored fecal, e.g. during transplanting, outside the storage unit. In some embodiments, the same pump is used for extracting and for transplanting processed fecal.

According to some embodiments, the storage unit includes one or more indicators to indicate a state of the processes fecal. In some embodiments, at least one of the indicators is a temperature indicator.

An aspect of some embodiments of the invention relates to a preparation of FT in a container having more than one compartment.

In some embodiments, in each compartment, fecal materials may be processed in different preservation conditions (e.g., gas composition, temperature, and/or liquid medium compositing) in accordance with the stage of FT preparation procedure.

For example processing the fecal materials in the presence of a liquid composition, having a desired viscosity in one compartment, may reduce the degradation of microorganisms resulted from a mechanical damage which may be applied during some stages of the processing, while filtering the fecal materials in a different compartment that contains a different liquid composition, may be favorable for a prolonged period of preservation.

In some embodiments, fecal materials are submerged in liquid medium in one compartment and refined from the liquid medium in another compartment. In another example, fecal materials are placed in low-oxygen atmosphere conditions at one compartment and at an ambient atmosphere conditions in another compartment.

An aspect of some embodiments of the invention relates to a preparation of FT in at least two separated compartments (e.g., a processing compartment and a filtering compartment).

In some embodiments, the preparation of FT includes fecal particles size reduction inside a processing compartment and filtering inside a filtering compartment.

In some embodiments, the two compartments are separated from each other. In some embodiments, the separation is a mechanical separation e.g., by a separating surface positioned between the compartments.

In some embodiments, the separation surface is a movable separating screen. Optionally, the separation is selective, which allows a transition of particles between the compartments based on their size.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary FT Fecal Processing Method

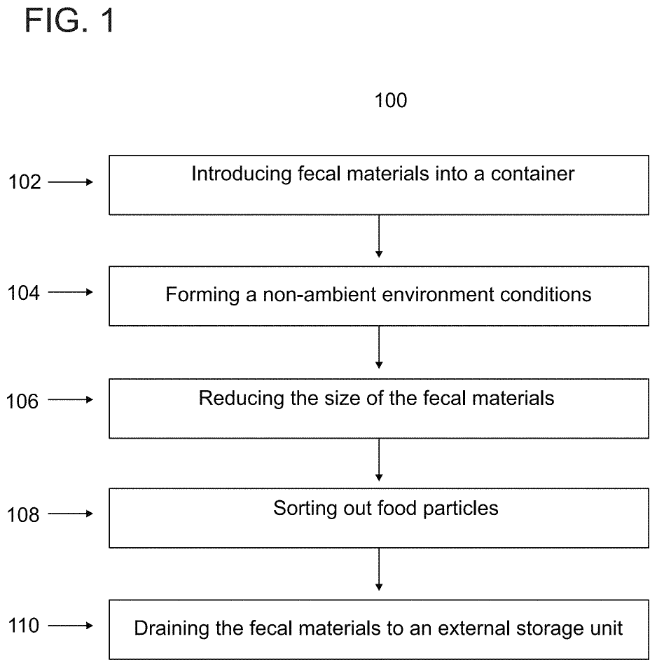

FIG. 1 shows a flow chart of a process 100 of preparation of fecal transplant, in accordance with some embodiments of the invention.

In some embodiments of the invention, fecal materials are processed under a desired environmental conditions which may have an advantage of reducing degradation of at least some sensitive microorganisms.

In some embodiments, the environmental conditions include using a low concentration of oxygen. Optionally or additionally, the environmental conditions comprise using a supportive liquid and/or exposing fecal materials to the desired temperature.

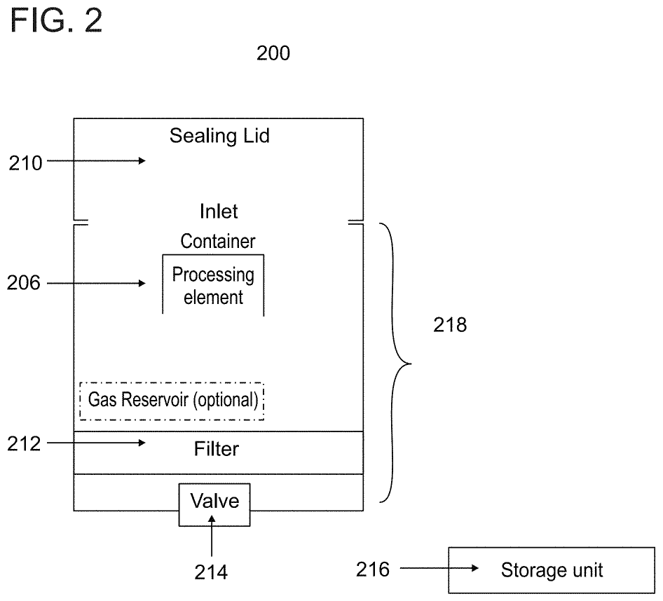

FIG. 2 shows a block diagram of a device 200 for preparation of fecal transplant for use in accordance with some embodiments of the invention, for example for use with the method of FIG. 1.

As shown at FIG. 2, fecal processing device 200 includes a processing container 218, and an optional sealing lid 210, and, optionally, an external storage unit 216.

Referring back to FIG. 1.

At 102, fecal materials are introduced into container 218.

Various methods of introduction may be used. In one example, a user defecates directly into container 218 (e.g., gravity based). In another example, the user manually collects feces (e.g., from a toilet bowl) and places them in container 218. In another example, feces are manually extracted from the user's rectum and/or colon, for example using an enema and then fed into container 218. In some embodiments, fecal materials are introduced into container 218 by using a valve or through other opening in container 218.

At 104, supportive conditions are optionally provided in container 218.

Supportive environmental conditions may affect the quality of the microbiota during the preparation and transplantation of fecal materials.

Typically, when preparing fecal transplants, the environments of fecal material changes from an inter-body intestinal environment to an external processing environment, to a storage environment, to a delivery environment and then back to an intra-body environment during transplantation.

These environments may be different from each other and/or one or more of these environments may have an effect on the micro biotic composition, and metabolic state when transplanted. For example, exposure to one or more of these environments may damage some of the species of microorganisms in the faces, and/or may lead to degradation of some of the species; For example, by reduction the amount of living bacteria and/or by alteration the ratio between the species in the fecal material, e.g., due to selective flourishing of certain species and death of certain species of microorganisms therein.

Another possible issue is contamination of fecal materials by microorganisms and/or chemicals which are not found in the original intestinal environment (e.g., due to a contact with urine and/or with the toilet bowl itself).

Another possible issue is the effect of the various environments on the non-micro biotic aspects of the transplant, for example, physical parameters of the feces such as the state of matter (i.e., solid and/or liquid) and/or texture of the feces. Such aspects may affect the effectiveness of the transplanting process itself, for example, the transplant delivery location, and/or effectiveness of micro biotic reception in the intestines.

In some embodiments of the invention, fecal materials are collected and/or processed and/or transplanted under controlled conditions that may allow maintaining a desired amount of bacteria and/or desired ratio between the bacterial species in the fecal materials.

In some embodiments, a desired texture and/or state of matter of the fecal transplant are maintained, optionally by processing the fecal materials to desired size and/or shape and/or texture.

Gas Introduction

In some embodiments, supportive environment conditions are formed, for example, by reducing the exposure of microorganisms to oxygen. Optionally, exposure is reduced by reducing the amount of ambient oxygen in container 218. In some embodiments, a low oxygen gas composition is introduced into the container right after sealing the container 218.

In some embodiments, user may introduce a desired gas composition into container 218. For example, prior to introducing fecal materials into the container and/or after and/or alongside introducing of fecal materials into the container.

In some embodiments, gas is introduced through an inlet positioned for example at the top section of container 218 (as shown in FIG. 2). Optionally, gas is introduced into the container via a valve and/or a tap (for example valve 214). Optionally or alternatively, gas is injected into the container.

In some embodiments, a reservoir, for example a bag containing desired gas composition is introduced into container 218. Optionally the bag may contain a mixture of different desired gases.

In some embodiments, the gas is released from the bag, upon activation of a processing element 206 which may have a cutting element. Optionally, such cutting element may cut the bag and release the gas from it.

Optionally, a gas reservoir is positioned in an inner lumen inside the walls of container 218. In some embodiments, the reservoir is separated from the inner lumen of container 218 by a separating membrane. Optionally, processing element 206 is operable to cut the membrane and release the gas from it.

Different bacterial groups may require different environmental conditions in order to survive. As such, in order to preserve the richness of the fecal microbiome it may be desirable to expose the bacteria to various environmental conditions, which include for example an anaerobic condition (for example to an atmosphere without unbound oxygen).

Optionally, exposure of microbiota to a microaerophilic condition (i.e., a gas composition having a concentration of oxygen which is lower than 18% and/or between 2% to 10%) is provided.

Optionally or alternatively, exposure of microbiota to an aerobic condition is provided.

In some embodiments, a gas composition comprising a mixture of gases is introduced into container 218 by a syringe. For example N₂—80%, CO₂—10%, H₂—5%.

In some embodiments, ambient oxygen is reduced by introducing a low oxygen gas composition into container 218, for example, before, during and/or after fecal material introduction. Optionally, this introduced gas composition comprising an oxygen concentration of at most 10%. Alternatively, a gas composition having a concentration between 3% to 18% of oxygen is introduced. Optionally or alternatively, a gas composition having a concentration at most 18% of oxygen is introduced.

In some embodiments, a gas composition having a concentration between 1% to 10% of carbon dioxide is introduced into container 218. Optionally or alternatively, a gas composition having a concentration of at most 10% of carbon dioxide is introduced.

In some embodiments, a gas composition having a concentration between 5% to 10% of hydrogen is introduced into container 218. Optionally a gas composition having a concentration of at most 5% of hydrogen is introduced. Optionally or alternatively, a gas composition having a concentration at most 10% of hydrogen is introduced.

In some embodiments, a gas pressure within container 218 is modified. For example, gas pressure is modified to be at most 1 atm. Optionally, or alternatively, the gas pressure is modified to be at least 1 atm.

In some embodiments, exposure to oxygen is diminished by reducing the contact between feces and the surrounding atmospheric gas for example by introducing a liquid into container 218 to form a barrier between submerged fecal materials and the surrounding gas atmosphere.

Optionally or additionally, exposure to oxygen is reduced by shortening the duration of conveyance of feces from a donor to processing device 200 during natural defecation of fecal materials, for example by placing container 218 inside a toilet bowl for immediate collection of fecal materials from the donor during natural defecation.

In some embodiments, exposure to some un-desired gases is reduced, by collecting feces directly from the donor's intestine.

In some embodiments, isolation between the inner environment conditions of processing device 200 and the outer environment is formed; optionally by sealing container 218 with sealing lid 210. A potential advantage of sealing container 218 is preventing a transition of un-controlled gases and/or liquids and/or solids into and out from container 218. For example, bad odors and/or fecal materials.

In some embodiments, the liquid is introduced into container 218 prior and/or alongside the introduction of the fecal materials. Optionally or additionally, the liquid is introduced after processing fecal materials and along with transplanting fecal materials to a recipient.

In some embodiments, such liquid introduction is performed by pouring a desired liquid into the container, optionally through the inlet, prior to sealing the container. Optionally or alternatively, the introduction is performed by injection of the liquid through the wall of container 218 and/or through sealing lid 210 by a syringe. Optionally or alternatively, the liquid introduction is performed by injection of the liquid through a valve, for example through valve 214.

In some embodiments of the invention, a liquid that contains supportive and/or preservative agents such as glycerol and/or saline is introduced. Optionally, a supportive liquid solution containing at most 30% volume of glycerol is introduced into container 218. Alternatively, a supportive solution having at least 10% volume of glycerol is introduced into container 218.

Optionally, a supportive solution having between 10% to 30% volume of glycerol is introduced into container 218.

In some embodiments of the invention, a liquid that contains supportive and/or preservative agents such as glucose and/or sucrose and/or polyethylene glycol is introduced. Optionally, a supportive liquid solution containing at most 10% volume of glucose is introduced into container 218. Optionally, a supportive liquid solution containing at most 10% volume of sucrose is introduced into container 218.

Optionally, a supportive liquid solution containing at most 10% volume of polyethylene glycol is introduced into container 218.

In some embodiments of the invention, the preservative liquid serves as a cryopreservative liquid medium, which may prevent the death of at least some species of the microbiota during freezing and/or during thawing.

In some embodiments, the temperature of the microorganisms is modified, optionally by modifying the temperature of the surrounding environment. For example, during the introduction of fecal materials into container 218, a cold liquid medium is also introduced into the container.

In some embodiments of the invention, the temperature is modified to be at most 25° C. In another example the temperature is modified to be between 0° C. to 25° C. In another example the temperature is modified to be between 4° C. to 25° C.

At 106, in some embodiments of the invention, the maximal dimension of at least some of the fecal materials is reduced. In some embodiments, the maximal dimension comprises one or more of a length of the fecal particles, a width of the fecal particles and/or an extent of the fecal particles.

Optionally, a maximal dimension of the particles is reduced to a range between 1 mm to 8 mm, for example to a range between 1 to 7 mm, 2 to 6 mm, 3 to 8 mm or any intermediate, smaller or larger value or range of values.

Typically, as the size of fecal materials reduces, the ratio between the inner section and the outer surface area of the fecal material changes, and more microorganisms, which are normally positioned in the depth of the fecal materials, is exposed to the external environmental conditions.

Further, more microorganisms become accessible and can pass from the fecal material to the surrounding liquid and/or gas environment.

In some embodiments, fecal materials are reduced in size to smaller fecal parts, optionally by a processing element 206 as exemplified in FIG. 2. Optionally fecal materials are reduced in size by applying mechanical force against the introduced feces.

In some embodiments, processing element 206 may include a size reducer, the size reducer includes a cutting element, for example, a cutting blade. Optionally or additionally processing element 206 may include a puncturing element by which fecal materials' inner sections are exposed to the surrounding environment, for example, a rod-like element

In some embodiments of the invention, the inner sections of the fecal materials are exposed, optionally by blending the fecal materials inside container 218, for example by a rotating element which may allow casting the fecal materials against the walls of container 218. Optionally or alternatively, the exposure is performed by shaking the container. Optionally or alternatively, the exposure is performed by milling the fecal materials, for example by a milling element. Optionally or alternatively, the exposure is performed by grinding the fecal materials.

In some embodiments, fecal materials are dispersed to form a colloid of a plurality of smaller parts of fecal materials inside container 218, optionally by using a dispersion element. For example, a rotating impeller by which rotational motion may be converted to vertexes or turbulent motion, operable to disperse feces within container 218.

At 108, in some embodiments of the invention, undigested food particles are separated out from the fecal material. This separation allows receiving relatively high content of microorganisms in the resulted FT.

In some embodiments, separation is performed by conveying fecal materials through a filter 212. Optionally fecal materials are conveyed gravitationally and/or by pressing the fecal materials through filter 212.

At 110, according to some embodiments of the invention, fecal are removed from container 218. For example, fecal materials are removed by draining the fecal materials through a valve 214. Optionally valve 214 is a uni-directional valve through which fecal materials and/or liquids and/or gases are drained from the inner section of container 218. Optionally valve 214 is a bi-directional valve, operable to allow a transition of liquids and/or solids and/or gases through it on both directions. Optionally the valve is configured to allow transition of fecal materials from the inner section of container 218 and to allow a transition of preservative liquids and/or desired gas composition from the outer environment into the inner section of container 218.

In some embodiments of the invention, valve 214 is positioned at the wall of processing container 218 and in some other examples, valve 214 is positioned at the base of the processing container 218.

Optionally, fecal materials are removed from container 218, for example by sucking the materials from the container by using a syringe. Optionally or alternatively, by pouring the fecal materials and/or liquids through a valve or through a tap.

In some embodiments, fecal materials are removed to a sealed vessel, for example to a storage unit 216.

In some embodiments, the storage unit may be positioned externally to processing container 218. Optionally the storage unit is positioned inside processing container 218.

In some embodiments of the invention, unit 216 is a vessel having an inner lumen therein, e.g. a capsule, a pill, and/or an enema bag.

In some embodiments, storage unit 216 is made of an elastic material.

In some embodiments of the invention, storage unit 216 has a tubular section and a wide section. In some embodiments, the tubular section has a smaller diameter than the wide section. In some embodiments, the tubular section is adapted to connect valve 214 and further adapted to be inserted into a user's anus.

In some embodiments of the invention, fecal materials are removed from container 218 directly to a recipient without being exposed to the outer environment conditions optionally directly to a recipient's intestine.

Direct conveyance of the fecal materials is performed optionally by removing the fecal transplant from container 218, directly to storage unit 216. For example, by connecting the tubular section of storage unit 216 to valve 214 and collecting the fecal transplant directly from container 218, insert the tubular section into the recipient's rectum, and squeeze the wide section of the storage unit.

According to some embodiments, storage unit 216 includes a manual pump to apply a positive pressure at valve 214, between the processing device and storage unit 216. In some embodiments, storage unit 216 is functionally similar to a syringe. In some embodiments, the storage unit 216 has a plunger to generate the pressure.

Optionally or alternatively, insertion of fecal transplant into the recipient body is performed by pills and/or capsules, which may store the fecal materials and can be administered by the user orally. In some embodiments, fecal materials are sucked from container 218 by a syringe and injected directly into the body of a recipient, for example, to the intestine.

### Exemplary Processing Method and Device Operation

In some embodiments of the invention, a donor of fecal materials may be a healthy individual, having a microbiota composition, which is suitable for transplantation. Optionally, fecal materials of a donor are pre-evaluated, prior transplantation, in order to determine the quality of a donor's fecal transplant. For example, such evaluation is based on a previous administration of the transplant, either by the donor himself or by another person.

In some embodiments, such pre-evaluation may allow determining a recommended administration regime.

In some embodiments, the donor, pre-donating a fecal sample, may administer laxative materials to induce diarrhea or other materials to induce constipation. Such materials may modify the liquids content in the donated fecal materials according to a desired processing procedure. For example, when a quick processing procedure is required, user may administer laxative materials to induce diarrhea to allow shortening the processing stage (e.g., the fecal processing stage).

In some embodiments, microbiota may be collected from various internal areas of the digestive system. For example, microbiota may be extracted from saliva, stomach juices, bile and/or pancreas secretions. Optionally, microbiota is extracted from the large and/or from the small intestine.

In some embodiments, the recipient is a patient, having a disordered intestine activity and/or having an intestinal disease. Optionally or alternatively, in cases when a medical procedure is predicated to harm self-intestinal microbiota, the ‘recipient’ is also the ‘donor’, donating microbiota for himself, namely prior preforming the medical procedure, in that case, FT is collected and stored for a later use.

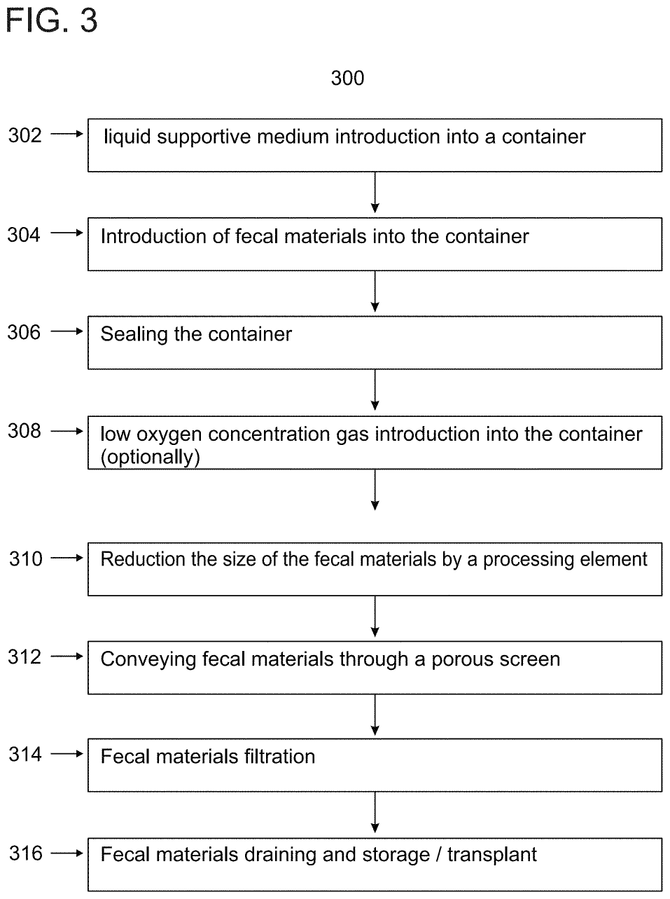

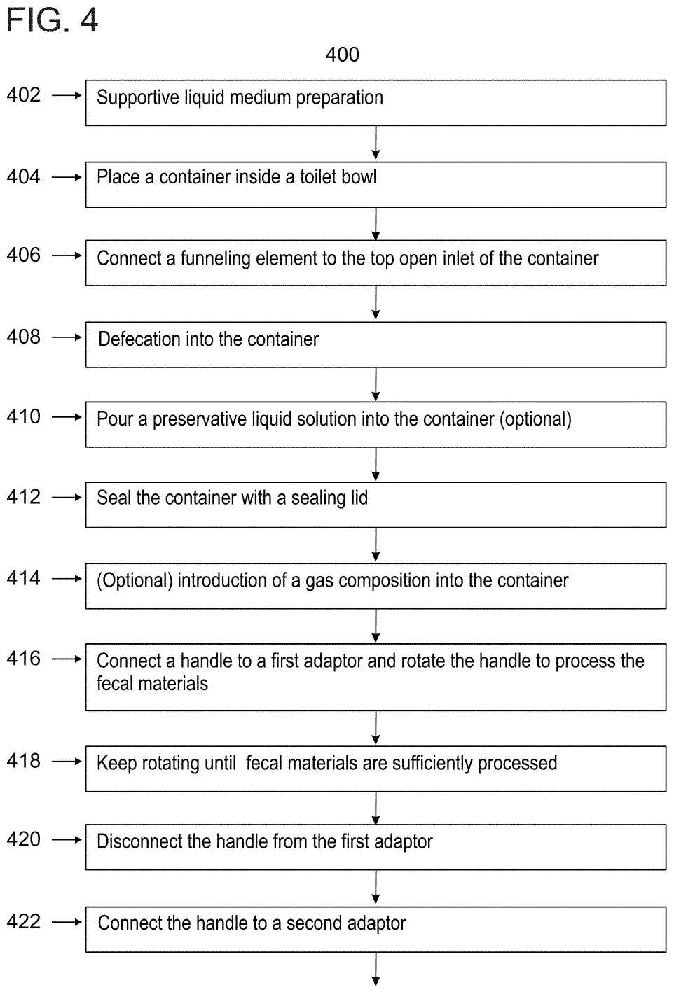

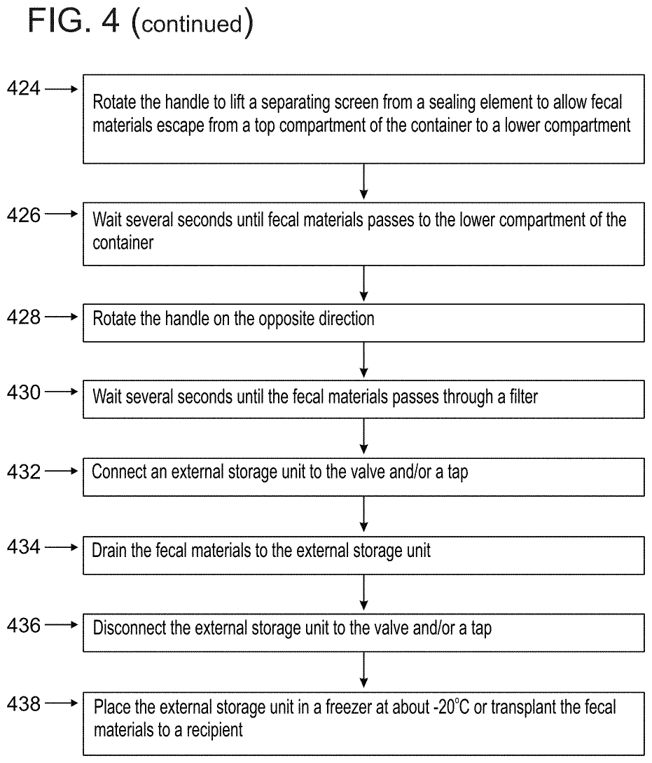

FIGS. 3 and 4 show, in a non-limiting manner, a general illustration of a processing device operation method (300) and exemplary actions taken by a user (400) for the preparation of a FT, in accordance with some embodiments of the invention.

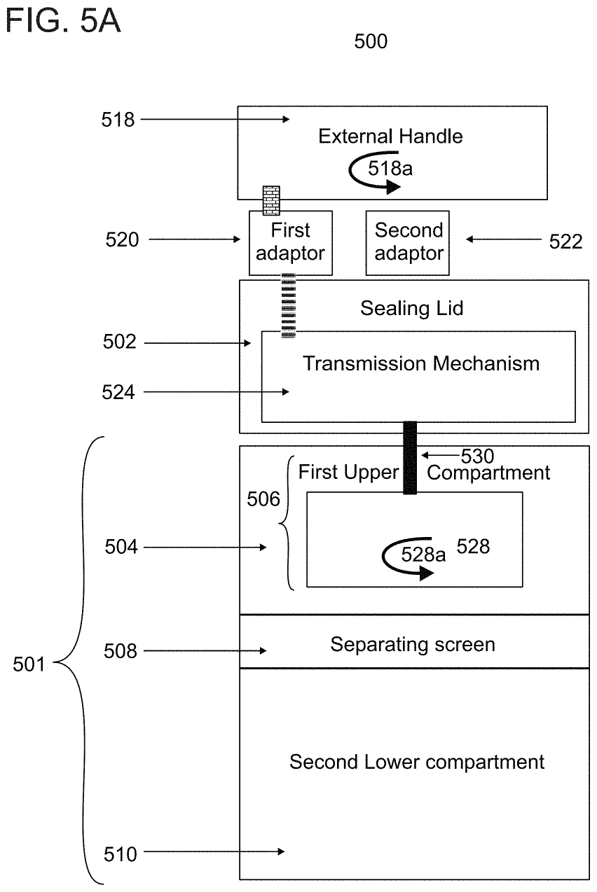

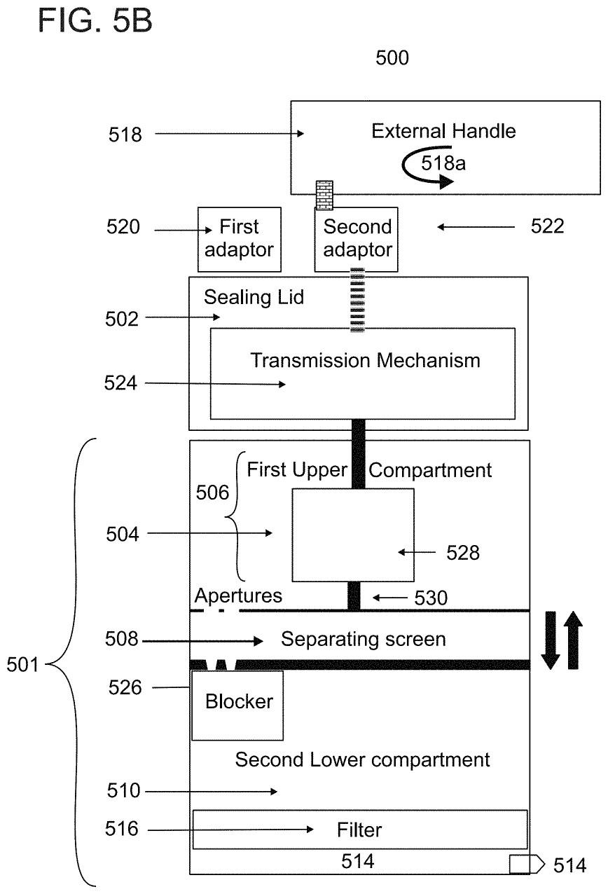

FIGS. 5A and 5B show exemplary embodiments of the invention, illustrating a block diagram of an exemplary fecal processing device 500 for preparation of fecal transplant for use in accordance with the methods of FIGS. 3 and 4.

At 302, a liquid supportive medium is optionally prepared and introduced into container 501.

At 402, in preparing the liquid medium, the user may modify one or more parameters of the introduced liquid (e.g., temperature, composition, state of matter).

Temperature Modification

As fecal materials contain different species of bacteria, wherein each species may have a different optimal growth temperature range, modifying the temperature of the liquid medium to desired temperature, may contribute to flourishing of some species and/or may prevent other species from thriving.

In some embodiments, a user may modify the temperature of the liquid medium, prior introduction the medium into the fecal processing container.

In some embodiments, the temperature is modified by cooling the liquid medium, optionally by using a cooling device (e.g., by a refrigerator). In another example, the liquid medium is modified, optionally by heating the liquid medium by a heating device (e.g., by a heater). For example, the user may modify the temperature of the liquid medium to below 20° C. to allow at least some of the microorganisms to flourish. In another example, the temperature may be modified to below 0° C. In another example, temperature is cooled to about 4° C. In some embodiments, the temperature is modified to a range between 0° C. to 20° C.

Supportive Agents

In some embodiments, the liquid medium may contain supportive agents. For example, the user may introduce into container 501 an ‘off the shelf’ prepared liquid solution that may contain preservatives. Optionally or alternatively, the user may introduce into container 501 a water and/or a saline-based liquid, and add preservative agents, such as glycerol.

Optionally liquid medium may contain between 10-30% glycerol. Optionally or alternatively, the liquid medium may contain between 10-20% glycerol. Optionally or alternatively, the liquid medium contains between 15-20% glycerol or any intermediate, smaller or larger value or range of values of concentrations.

In another example, the user may alter the ratio between agents within the introduced liquid medium. Optionally, the user may gradually introduce different types of preservatives into container 501.

In some embodiments, user may use a liquid preservative composition and pour it into the container.

Optionally or alternatively, user may mix several types of preservative liquids and pour them into container 501.

Sterilization

In some embodiments, a liquid medium is sterilized, prior introduction into container 501. In some embodiments, such sterilization is performed for example by autoclave or by UC. Optionally, the sterilization is performed by combinations thereof.

In some embodiments, a sterile liquid medium is introduced into container 501. Optionally, about 300 cc of sterile saline and about 100 cc of sterile glycerol are added.

In some embodiments, a pre-sterilized liquid is introduced into the container.

At 304, in some embodiments, fecal materials are collected from a donor and introduced into container 501. Optionally, fecal materials are introduced to a container which may include the supportive liquid medium. In some embodiments, fecal materials are introduced into the container prior to introduction of the liquid medium.

As exemplified at 404, the user may introduce fecal materials directly into container 501, optionally by placing container 501 inside a toilet bowl and defecate into it.

In some embodiments, as presented at 406, the user may position a funneling element (not shown) onto a toilet bowl perimeter and connect the funneling element to the container 501 which is placed in the toilet bowl and under the element.

After sitting on a seat of a toilet bowl, the user may naturally defecate towards the funneling element, to allow fecal materials a continuous transition along the funneling element and slide towards the processing container.

A potential advantage of directing fecal materials immediately to the processing container is reducing the duration of fecal exposure to contaminations and/or to various undesired liquid and/or atmospheric conditions.

At 306, optionally after fecal materials are introduced into container 501, the container is sealed by a sealing lid 502.

At 308, optionally the gas atmosphere composition inside the container 501 is modified. For example, at 414 user may introduce a gas composition comprising at most 18% of oxygen and/or at most 10% of carbon dioxide and/or at most 5% of hydrogen and/or at most 80% of nitrogen.

In some embodiments, a user may modify the ratio between the gases in the introduced gas composition. Optionally, by introducing additional selected gas (e.g., oxygen and/or carbon dioxide and/or nitrogen and/or hydrogen).

In some embodiments, gas composition comprising an oxygen concentration between 3% to 18% is introduced into the container. For example oxygen concentration is between 3% to 18%, between 3% to 10%, between 3% to 8%, or any intermediate, smaller or larger value, or range of values of concentrations.

In some embodiments, gas composition comprising carbon dioxide concentration between 1% to 10% is introduced into the container. For example carbon dioxide concentration is between 2% to 8%, between 3% to 7%, between 5% to 10%, or any intermediate, smaller or larger value, or range of values of concentrations.

In some embodiments, gas composition comprising hydrogen concentration between 5% to 10% is introduced into the container. For example hydrogen concentration is between 5% to 8%, between 5% to 7%, between 6% to 10%, or any intermediate, smaller or larger value, or range of values of concentrations.

Optionally ambient air is introduced (e.g. air from the immediate surrounding environment).

At 310, the size of the fecal materials is reduced, optionally by a processing element 528. In some embodiments, the processing element 528 is a size reducer which includes for example a cutting element (e.g., a cutting blade).

In some embodiments, fecal materials are divided into a smaller parts having a maximal dimension which smaller than 8 mm. Optionally, a maximal dimension of the particles is reduced to a range between 0.1 mm to 8 mm, for example to a range between 0.1 to 7 mm, 2-6 mm, 3-8 mm or any intermediate, smaller or larger value, or range of values.

A potential advantage of reducing the size of the fecal particles is the enhancement of exposure of microorganisms positioned at the inner sections of the fecal particles to the surrounding supportive liquid medium, caused by the increased lack of sheltering induced by the divided fecal particles.

At 416, the user may activate processing element 528, by which the fecal materials are reduced in size. Optionally the processing element is manually activated by rotation of an external handle 518.

At 418, the user may apply rotation, for reducing the size of the fecal materials.

For example, rotation may last for less than 2 minutes. Optionally or alternatively, rotation may be performed intermittently.

In some embodiments, a homogenous colloid of fecal materials parts and suspension of microorganisms is produced, due to a centrifugal force applied by rotation of the processing element 528.

In some embodiments, fecal materials are dispersed in the liquid medium. Optionally, fecal materials are dispersed by blending the fecal materials in the container 501.

At 312, fecal materials are conveyed through a separating screen 508. In some embodiments, the screen allows transition of only small particles while detaining transition of a large particles and fecal clumps.

In some embodiments, the screen, having apertures therein, is positioned under the introduced fecal materials. Optionally, the apertures are selectively blocked by a blocker (see FIG. 5B, 526).

In some embodiments, the user may alter the position of screen 508 (e.g. elevate and/or lower and/or tilt and/or horizontally move the screen) in relation to the blocker 526 to allow opening of the apertures and to allow transition of fecal materials and/or liquids and/or gases through the apertures.

As presented at 420-426, the position of the screen is optionally modified by an elevation mechanism.

In some embodiments, the elevation mechanism is manually controlled by rotation of a rotating handle 518. For example, the rotating handle is configured to connect to an adaptor, which is operable to transfer rotation of handle 518 to a transmission 524. The transmission 524 mechanism is operable to rotate a lead screw 530, which is further configured to screw into the separation screen. As the lead screw 530 screws into the separation screen 508, the position of the screen is altered according to the handle's rotation direction.

In some embodiments, by rotating handle 518 for example clockwise, the separating screen 508 is elevated, and a gap between the separating screen and the blocker 526 is formed. As the gap expands, fecal materials are allowed to pass from an upper processing compartment 504 downwards to a filtering compartment 510.

In some embodiments, for example as presented at 428, a user may optionally rotate handle 518 for example counterclockwise to reverse the direction of movement of screen 508 and narrow a gap between the screen and the blocker 526.

As fluids and fecal materials transit to the lower compartment, lowering the separating screen both reduces further transition of fecal materials to the lower compartment and induces a positive pressure on the already conveyed liquids and fecal materials.

Optionally, the liquids and fecal materials are conveyed toward a filter 516.

At 430 in some embodiments, during conveyance of the fecal materials and/or liquids through the filter 516, solid particles larger than a filter's pores are sorted, while smaller particles of fecal materials and/or liquids, passes through the pores of the filter.

In some embodiments, the pores have a maximal extent which is smaller than 2 mm. Optionally, a maximal dimension of the pores is between 0.1 mm to 2 mm, for example to a range between 1 to 2 mm, 0.1 to 1.8 mm, 0.5 to 1.5 mm or any intermediate, smaller or larger value, or range of values.

In some embodiments, fecal materials are conveyed gravitationally. Optionally or alternatively, the user may activate a pressing element, for example, the base of the separating surface 508, by which fecal particles are mechanically pressed toward the filter.

At 316, fecal materials are optionally removed from the container.

At 432-436, in accordance with some embodiments, a user may store the fecal transplant in a storage vessel.

At 438, user may store the collected fecal materials in cold environmental conditions, for example at about −18° C.

FIGS. 5A and 5B further illustrate an exemplary embodiment 500 of a fecal processing device 500 in two operational modes according to some embodiments of the invention;

For example, one mode of operation includes activation of a fecal processing element by rotation of an external handle; and a second mode of operation includes initiation transition of fecal materials and/or liquids between compartments within the processing container; both initiated by rotation of the same handle, but possibly attached and/or operated differently.

FIG. 5A is an exemplary embodiment of the invention, illustrating an exemplary operation mode, that includes an operation of processing element 506 by rotation of external handle 518 (rotation is marked as a curved arrow inside external handle 518).

Rotation Transmission

In some embodiments, the external rotating handle 518 is operable to connect to an adaptor which is positioned on a sealing lid 502. In some embodiments, the handle 518 and the adaptor are shaped and sized to fit each other. Optionally or alternatively, a locking element operable to maintain the handle and the adaptor adjacent to each other is incorporated (e.g., a screw and a bolt).

In some embodiments, the rotating handle 518 and the adaptor are permanently connected to each other.

In some embodiments, sealing lid 502 includes two adaptors, a first adaptor 520 and a second adaptor 522, each one is operable to rotate clockwise and/or counterclockwise. Optionally, the user may connect handle 518 to any one of the adaptors and rotate handle 518.

In some embodiments of the invention, at least one of the adaptors is connected to a transmission 524. Optionally, the transmission is situated in a transmission compartment which is positioned in the sealing lid 502. The transmission compartment may include a top surface and a base surface, and a circumferential wall defined inside sealing lid 502.

In some embodiments, the first adaptor 520, and/or the second adaptor 522 has an external shaft (e.g., a shaft, pawl, and/or a bolt) projected outside from the top surface of the transmission compartment (shown in FIGS. 5A and 5B as a dotted line), operable to connect to external handle 518. Each adaptor may also include an inner shaft (shown in FIGS. 5A and 5B as a dashed line), positioned inside the transmission compartment, operable to connect transmission mechanism 524.

In some embodiments, rotation of the inner shaft leads to rotation of transmission 524.

In some embodiments, the transmission mechanism 524, is interconnected to processing element 506 via a lead screw 530 (shown in FIGS. 5A and 5B as a solid line), operable to transmit rotation of external handle 518 to processing element 506.

Optionally, the shaft (positioned between transmission mechanism 524 and the processing element 506), has a first end and a second end, optionally, the first end is connected to the transmission mechanism 524 and the second end is connected to at least one cutting element 528.

Processing by Rotation

In some embodiments, processing element 506 is operable to rotate the attached cutting element 528 is (in direction of arrow 528a) and reduce the size of the fecal material by cutting the fecal materials during the rotation.

Optionally, rotation of handle 528 leads to rotation of processing element 506. In some embodiments, rotation of the handle is performed manually and/or an automatically.

In some embodiments, processing device 500 includes a container 501 divided to at least two compartments (e.g., upper and lower) by separating screen 508 Wherein, the processing element 506, may be positioned at an upper compartment 504 and wherein the processing element 506 may be positioned at a second lower compartment 510.

Referring back to FIG. 5B;

FIG. 5B is an exemplary block diagram of processing device 500 that includes at least two compartments. In some embodiments, fecal materials are processed in one compartment and filtered in a second compartment. In some embodiments, fecal materials and/or fluids are conveyed between the compartments through a funneling path formed between the two compartments.

At FIG. 5B the two opposite arrows at the right side of the diagram show an exemplary movement of a separating screen 508 as an example of forming the path between compartments in the processing container 501, according to some embodiments of the invention.

Fecal Materials and/or Liquids Conveyance in the Processing Device

In some embodiments, a path between a first upper compartment 504 and a second lower compartment 510 is fully/partially blocked by a blocker 526. Optionally, the separator is perforated. In some embodiments, the perforations are blocked by blocker 526 positioned adjacently to the perforations. In some embodiments, the blocker 526 and the separating screen 508, positioned between the compartments.

In some embodiments, separating screen 508 has a plurality of apertures which are selectively blocked the blocker 526. The apertures are optionally blocked when blocker 526 and separation screen 508 contact each other. Optionally or alternatively, the apertures are opened when a gap is formed between blocker 526 and separation screen 508 and transition of fluids and/or fecal materials between upper compartment 504 and lower compartment 510 is enabled.

In some embodiments, a path between the two compartments is formed alongside separation screen 508. For example, the external perimeter of separating screen 508 may be smaller than the inner perimeter of processing container 501 allowing, fecal materials and/or liquids may flow from the upper compartment 504 to the lower compartment 510 along the sides of the screen 508. In some embodiments of the A potential advantage of preventing large fecal materials and/or large undigested solid particles transition from the processing compartment 504 to the filtering compartment 510 is the prevention of blockage the filter 516.

Further, elimination and/or reduction of large food particles away from the prepared fecal transplant may increase the quality of the transplant which is optionally defined by its homogeneity.

In some embodiments, separating screen 508 is operable to move by rotation of external handle 518 (in direction of arrow 518a).

In some embodiments of the invention, separating screen 508 is operable to move by rotation of a lead screw 530 (shown as a solid line). As the lead screw turns, for example clockwise, the screw is screwed into the separating screen 508 and separating screen 508 is either elevated or lowered (according to the direction of rotation of the external handle 518).

In some embodiments, user may rotate external handle 518 and lift up separating screen upwards, to allow fecal materials and fluids to pass through the pores of screen 508.

Optionally, after lifting the screen, user may reverse the rotation direction to lower back the separating screen 508.

In some embodiments of the invention, the position of separating screen 508 may affect the rate of transition of fecal materials and/or liquids from the top compartment 504 to the lower compartment 510. For example, as a gap between the separating surface 508 and blocker 526 is widened, a flux of fecal materials and/or liquids passes from the upper compartment 504 toward the lower compartment 510 increases.

A Gravitational Flow of Fecal Materials and/or Liquids Inside a Processing Device

In some embodiments, fecal materials may be introduced into a fecal processing device 500, conveyed through a separating screen 508, and filtered through a filter 516 which is optionally positioned at the base the fecal processing device 500. In some embodiments, filter 516 defines an upper border of a collection chamber 514.

In some embodiments, after fecal materials are filtered through filter 516 and conveyed to collection chamber 514, fecal materials may be conveyed through valve 518.

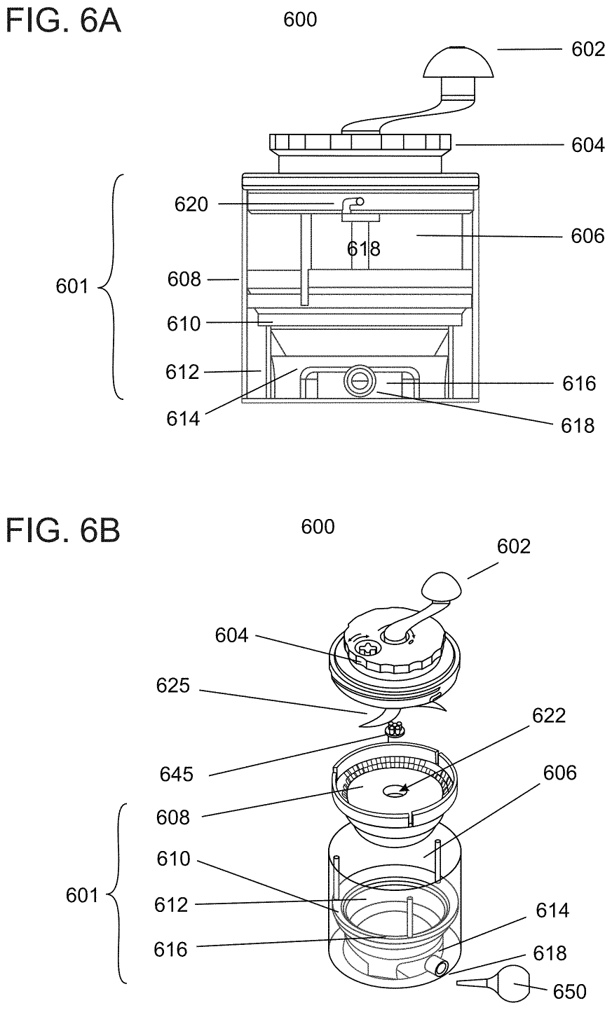

FIGS. 6A and 6B illustrate an exemplary embodiment of a perspective side view of a fecal processing device in an assembled configuration (FIG. 6A) and in a disassembled configuration (FIG. 6B).

Various optional features are illustrated at FIGS. 6A and 6B, which include an exemplary fecal processing device 600 having, for example, a top lid 604 operable to seal a processing container 601. Referring to FIG. 6A;

Securing Sealing Lid to a Processing Container

In some embodiments, processing container 601 is sealed, optionally with sealing lid 604. Optionally, the sealing is secured by inserting a pin into at least one securing pin groove 620 positioned at the perimeter of the lid, configured to secure sealing lid 604 to the processing container 601.

In some embodiments, user may insert a securing pin (e.g., a shaft, pawl, and/or a bolt) through the wall of the processing container 601 into securing pin groove 620. As sealing lid 604 is secured to processing container 601, rotation of the lid 604 is prevented.

For example in some embodiments, external rotating handle 602 and processing element 618 are interconnected to sealing lid 604 from opposite sides of sealing lid 604. When external handle rotates, the rotation is transmitted to a processing element 618 by a transmission positioned there between, which rotates accordingly. When secured, even when rotation is applied, the lid 604 remains fixed.

Orientation of the Compartments

In some embodiments, processing container 601 comprises an inner section, wherein fecal materials are processed wherein the inner section is divided into at least two compartments. For example, compartment 606 and compartment 612.

Optionally, the dimensions of the at least two compartments are modified, for example by altering the position of a separating screen 608 between them.

In some embodiments, the at least two compartments, are positioned one above the other.

Separation Screen and Blocker

Blocker

In some embodiments, separating screen 608 is operable to move in relation to a blocker, for example blocker 610, which blocks the movement of the screen 608.

For example, blocker 610 may be positioned under separating screen 608 and blocks descending of the screen under the position of the blocker 610. In another example, blocker 610 is positioned alongside separating screen 608 and blocks lateral movement of the screen in accordance with the position of blocker 610 (not shown).

In some embodiments of the invention, blocking element 610 is fixed to the inner wall of processing container 601. Optionally or alternatively, blocking element 610 is operable to move in processing container 601.

In some embodiments, blocker 610 has an external perimeter and an inner perimeter. The external perimeter of blocker 610 is shaped and seized in accordance with the inner perimeter of processing container 601. In some embodiments, when placed into processing container 601, the external perimeter of blocking element 610 is tightly adjacent to the inner perimeter of the wall of the processing container 601.

In some embodiments, a gap between the wall of processing container 601 and the external perimeter of blocker 610 is minimized to prevent the transition of gases and/or liquids and/or solids between them.

In some embodiments, blocker 610 inner perimeter has a funneled geometry (e.g., a cylinder and/or a cone shape) to allow conveyance of fluids and fecal materials gravitationally from an upper compartment 606 to a lower compartment 612 of processing container 601.

Separation Screen

In some embodiments, separating screen 608 is a sealed surface. Optionally, the sealed surface is positioned between the upper and lower compartments.

Optionally or alternatively, at least some area separation screen 608 is perforated.

In some embodiments, separation screen 608 is flexible, operable to adjust its shape in accordance with the weight of the fecal materials and/or liquids placed there onto.

Collection Compartment

In some embodiments, fecal materials, and/or liquids are filtered by a filter 616 and conveyed to a collection compartment 614.

In some embodiments, collection compartment 614 is positioned under lower compartment 612.

In some embodiments of the invention, collection compartment 614 is defined by an upper wall (which is optionally filter 616), a base, and a circumferential side wall. Optionally, the base of collection compartment 614 is defined by the base of processing container 601. In some embodiments, fecal materials and/or fluids and/or gases are conveyed gravitationally from the filter and accumulated in the inner section of collecting compartment 614.

Dimensions

In some embodiments, a portable fecal processing container which also sized and shaped to fit into a toilet bowl is provided. Such a portable device has small dimensions that allow using the device at home without being restricted to a laboratory use only.

A potential advantage of a portable device, is enabling a user to prepare a fecal transplant for self-use at home.

In some embodiments, the portable device may be inserted to a sterilization device as a “one piece” while the fecal materials remain inside the container in sealed environment conditions.

In some embodiments, fecal processing container 600 has an external height and diameter which are smaller and/or equal than the dimensions of a standard toilet bowl. For example, the height of processing container 600 is smaller than and/or equal to 150 mm. Optionally, the height of processing container 600 is in a range between 145 mm to 120 mm, for example in a range between 130 to 120 mm, 135-125 mm, 120-125 mm or any intermediate, smaller or larger value, or range of values.

For example, the diameter of processing container 600 is smaller than and/or equal to 200 mm.

Optionally, the diameter of processing container 600 is in a range between 150 mm to 200 mm, for example in a range between 160 to 190 mm, 150-160 mm, 150-155 mm or any intermediate, smaller or larger value, or range of values.

In another example, the inner volume of processing container 600, defined by the internal diameter between the side walls of container 600, its top outlet, and its base, is smaller than and/or equal to 1.4 L.

Optionally, the inner volume of processing container 600 is in a range between 1 L to 1.4 L, for example in a range between 0.5 L to 1.4 L, 1.2 L to 1.4 L or any intermediate, smaller or larger value, or range of values.

In some embodiments, fecal processing container 600 is portable, having a total weight that allows an average user to move the device without using any external accessories. For example, the weight of processing container 600 is smaller than and/or equal to 2 Kg.

Optionally, the weight processing container 600 is in a range between 0.5 Kg to 2 Kg, for example in a range between 0.5 Kg to 1.8 Kg, 0.8 Kg to 2 Kg, 0.9 Kg to 1.9 Kg or any intermediate, smaller or larger value, or range of values.

Fecal Materials and/or Liquids Removal from the Processing Device

In some embodiments, fecal materials and/or fluids are removed from the inner section of the collection compartment 614. For example, by an orifice positioned at the wall and/or at the base of the compartment. In another example, fecal materials and/or fluids are removed by valve 618 and/or a tap, optionally positioned on the wall of the collecting compartment 614.

Referring back to FIG. 6B;

FIG. 6B illustrates an exemplary embodiment of a perspective view of fecal processing device 600 in a disassembled configuration.

Exemplary Parts Arrangement

Device Positioning in a Toilet Bowl:

In some embodiments, processing container 601 has a flat base configured to be positioned stably inside a toilet bowl or onto a solid stable surface.

Separating Screen

In some embodiments of the invention, prior to introducing fecal materials into processing container 601, separating screen 608 is inserted into processing container 601 and positioned onto blocker 610. Optionally, separating screen is secured to blocker 610 (e.g., by a screw and/or a pin and/or a gluing element).

In some embodiments, separating screen 608 comprises a plurality of apertures. The aperture may have various sizes and shapes.

For example, the aperture may have a maximal diameter in a range between 1 mm to 8 mm. Optionally, a maximal diameter of the apertures is between 2 mm to 8 mm, for example in a range between 3 to 8 mm, 2-6 mm, 3-7 mm or any intermediate, smaller or larger value, or range of values.

As separating screen 608 is placed onto blocker 610, the apertures are sealed by blocker 610 and transition of fecal materials and/or liquids from the upper compartment 606 to the lower compartment 612 is prevented.

Sealing Lid

In some embodiments of the invention, sealing lid 604 is positioned onto a top inlet of processing container 601. For example, sealing lid 604 is placed onto processing container 601. Optionally sealing lid 604 is secured to processing container 601 (e.g., by a securing pin and/or a screw and/or a pin and/or a gluing material).

In some embodiments, sealing lid 604 and processing element 625 are connected to each other and placed together inside processing container 601 as a united assembly.

Valve/Tap

In some embodiments, a valve and/or a tap is positioned at the wall of processing container 601. For example, a valve is positioned at the peripheral wall of the container. Optionally, a valve is located at the base of the container.

In some embodiments, processing container 601 includes more than a single valve and/or a tap.

Optionally, the valve is a tap, having two modes of operation, a closed, and an open mode.

Storage Unit

In some embodiments of the invention, after fecal materials are introduced into processing container 601 and processed, fecal materials are filtered and removed from the container. In some embodiments, fecal materials are removed from processing container to an external storage unit 650.

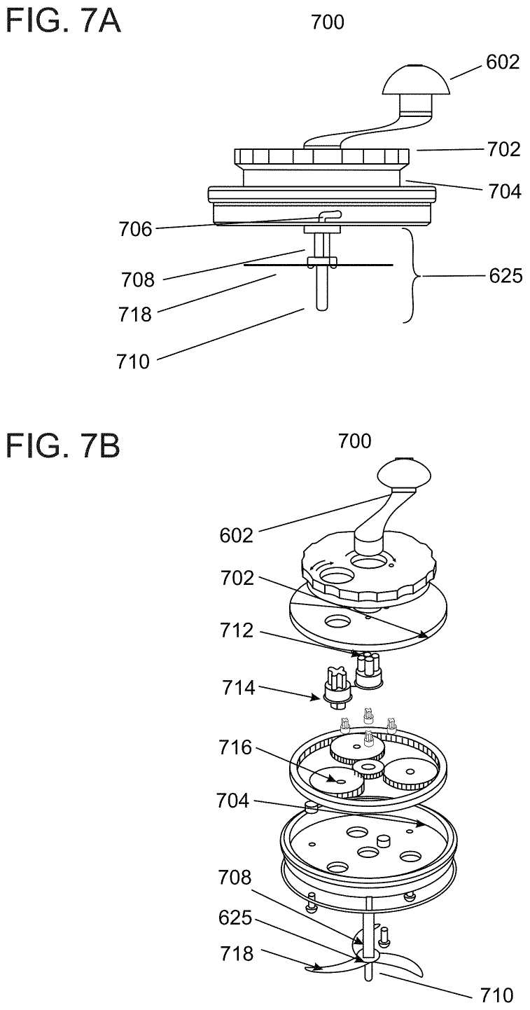

FIGS. 7A and 7B illustrate an exemplary embodiment of a perspective side view of a sealing lid 700 in an assembled configuration (FIG. 7A) and in a disassembled configuration (FIG. 7B).

Various optional features are illustrated in FIGS. 7A and 7B, which include an exemplary sealing lid having for example, a top surface, a bottom surface, a circular perimeter, and a securing element.

Referring to FIG. 7A;

Sealing Lid as a United Assembly

In some embodiments, a sealing lid 700 is an assembly of an operation element, a transmission element, and a processing element, gathered as a united piece.

In some embodiments, the operation element is an external handle 602. In another example, the operation element is a motor, adapted to propel manually and/or automatically a transmission element.

In some embodiments, the transmission element is a transmission mechanism adapted to transmit operation initiated by the operation element to a processing element. In some embodiments, the transmission element comprises a transmission gear 716. Optionally, the transmission element comprises a piston, operable to transmit operation initiated by the operation element to the processing element 625.

External Handle

In some embodiments, an external handle 602 is adapted to connect and/or disconnect to a top surface 702 of sealing lid 700 via an adaptor.

Transmission Compartment

In some embodiments, a transmission compartment is positioned inside the sealing lid 700. In some embodiments, the transmission compartment is defined between a first top surface 702 of sealing lid 700 and a second bottom surface 704.

Optionally, the compartment is situated in an inner section of sealing lid 700 which is defined by the inner perimeter of the walls of the lid.

Processing Element

In some embodiments of the invention, a processing element 625 is connected to sealing lid 700. Optionally, processing element 625 is operable to rotate.

In some embodiments, processing element 625 comprises a shaft 708 having a first end and a second end, the first end is connected to the bottom surface 704 of sealing lid 700, and the second end is operably connected to separating screen (as shown for example at FIG. 6B element 608).

In some embodiments, shaft 708 is connected to a size reducer, for example, blade 718. size reducer may include more than one blade.

In some embodiments, the blade of the size reducer is sharp. Alternatively, the blade of the size reducer is blunt.

In some embodiments, the length of and width of size reducer 718 are equal. Optionally or alternatively, the length of and height of size reducer 718 are equal. Optionally or alternatively, the width of and height of size reducer 718 are equal. In some embodiments, size reducer 718 is curved.

Gripping of Separating Screen

In some embodiments, shaft 708 includes a gripping element, for example a threaded screw 710, for holding separating screen (as shown for example at FIG. 6B element 608). Optionally, the gripping element is a hook and/or a magnet and/or a tether adapted to connect separating screen 608.

In some embodiments, threaded screw tip 710 is screwed into separating screen by rotation of external handle 602 and rotation of shaft 708.

Exemplary Sealing Lid Dimensions

In some embodiments of the invention, sealing lid 700 is sized and shaped to seal processing container (for example processing container 601 at FIG. 6A). Sealing lid 700 is portable having a size and a weight which are suitable to allow an average user to move the lid without using any external accessories.

Referring to FIG. 7B;

Parts Arrangement

In some embodiments, external handle 602 is connected to lid 700 by at least one adaptor and by rotation of handle 602 transmission mechanism 716 which is positioned inside lid 700, rotates accordingly.

Optionally, activation of transmission mechanism 716 activates the rotation of shaft 708 and rotation of size reducer 718. Optionally, activation of transmission mechanism 716 activates rotation of threaded tip 710.

External Handle Adaptor

In some embodiments, external handle 602 is connected to a top surface 702 by an adaptor, embedded in the top surface, which is further connected to transmission 716. In some embodiments, at least two adaptors are embedded in top surface 702. Optionally, the at least two adaptors are connected to transmission 716.

In some embodiments of the invention, external handle 602 has two operation modes;

A first mode, by which processing element (shown at FIG. 6B element 625) is activated; and

A second mode, by which movement of separation screen 608 (shown at FIG. 6B element 608) is activated.

Optionally, each operation mode is controlled by a different adaptor. For example, by connecting handle 602 to a first adaptor and rotating the handle, the rotation is transmitted by a transmission gear to processing element 625. In another example by connecting handle 602 to the second adaptor and rotating the handle, the rotation is transmitted by transmission gear to shaft 708 and to threaded tip 710, to allow elevation or lowering separation screen 608.

Transmission Compartment

In some embodiments, sealing lid 700 comprises a transmission compartment defined by an inner section of the lid. For example, the transmission compartment is defined by the top surface 702, second the bottom surface 704 of lid 700, and its perimeter.

In some embodiments, transmission compartment is a sealed compartment, having adaptors (each having a shaft) that project from the compartment through orifices and adapted to connect to handle 602.

Transmission

In some embodiments, sealing lid 700 accommodates a transmission mechanism comprises transmission gears situate horizontally, one in respect to the other, and fixed by the borders of the transmission compartment.

Optionally or alternatively, sealing lid 700 accommodates a transmission mechanism comprises an assembly of reel, and a rope wound around the reel. The rope further having a free edge.

In some embodiments, the reel is positioned horizontally inside sealing lid 700 and having a centered rotation axle.

The reel is further held by a spring, which is positioned inside the lid.

In some embodiments, the reel is configured to connect processing element 625 via shaft 708 which is connected to the rotation axle.

In some embodiments, user may pull the rope to allow rotation of the reel and the rotation of processing element 625.

Optionally or alternatively, rotation is achieved by connecting the processing element 625 to an external motor.

Processing Element Orifice

In some embodiments, sealing lid 700 comprises an orifice at the bottom surface 704 through which shaft 708 projects. In some embodiments, the orifice comprises an isolation agent (e.g., a glue, a gasket) positioned between the inner perimeter of the orifice and the projected shaft 708, that prevents transition of gases and/or liquids and/or solids between the transmission compartment and the processing container (element 601 at FIG. 6A).

Processing Element

In some embodiments of the invention, there is provided a processing element 625 which allows reducing the size of introduced fecal materials.

In some embodiments, transmission 716 is interconnected to processing element via shaft 708.

Optionally, transmission 716 is configured to rotate the processing element.

In some embodiments, the processing element 625 includes shaft 708 and a size reducing element 718. Optionally, the size reducing element 718 includes at least one cutting blade. A size reduction is achieved by cutting fecal materials upon rotation of processing element 625 in proximity to fecal materials.

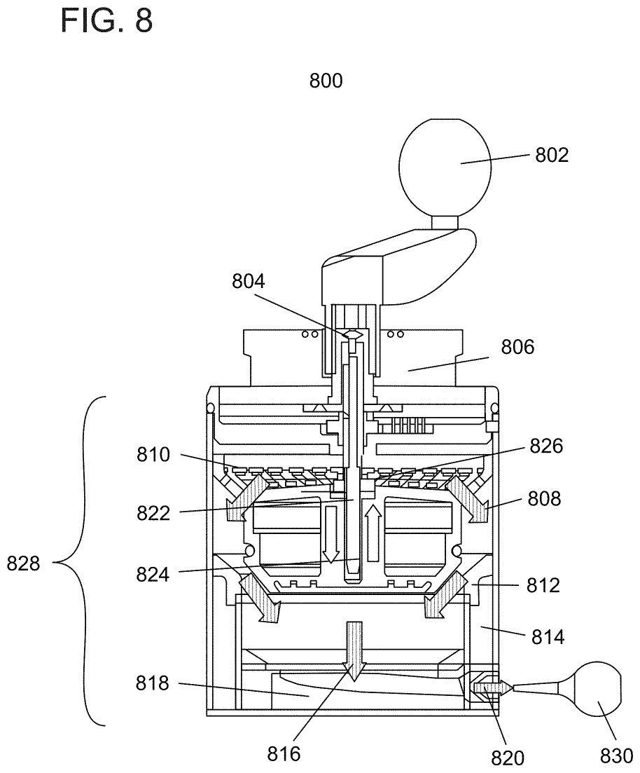

FIG. 8 is a side cross-sectional view of the fecal processing device.

FIG. 8 illustrates the relation between parts in a fecal processing device 800, and the manner by which some parts move with respect to each other. FIG. 8 also illustrates the manner of transition of fecal materials in the device, for example from an upper compartment to a lower compartment and/or from the inner section of the fecal processing device to an external storage unit, according to some embodiments of the invention.

In some embodiments, an external handle 802 is interconnected via an adaptor 804 to a sealing lid 806. A transmission positioned inside lid 806 is interconnected to a proximal end of a shaft 822 (in respect to lid 806). The shaft is connected to a separation screen 810 via a threaded screw 824, positioned at its distal end, which is operable to be inserted into separation screen 810.

In some embodiments, rotation of external handle 802, rotates shaft 822 and allows insertion of threaded screw 824 into screen 810. As the threaded tip penetrates screen 810, the screen is elevated towards lid 806 (movement of separation screen 810 is shown as two opposite white arrows).

In some embodiments, when screen 810 is lifted towards lid 806, a gap is formed between separation screen 810 and a blocker 812.

In some embodiments, blocker 812 is not operable to move and lifting separation screen 810 forms a gap between the blocker and the separation screen 810 is formed.

Optionally or alternatively, separation screen 810 is not operable to move, and the blocker is operable to move. Optionally, lowering the position of blocker 812 may form a gap between blocker 812 and separation screen 810.

In some embodiments of the invention, separation screen comprises a first top surface and a second bottom basin. In some embodiments, the top surface has a larger diameter than the second bottom basin. In some embodiments, when separating 810 is positioned adjacently to blocker 812, the top surface of separation screen 810 is situated above blocker 812, for example at an upper compartment 808 of the processing device 800 while the second bottom basin is situated at a lower compartment 814.

Optionally or alternatively, when separating 810 is elevated towards sealing lid 806, the top surface of separation screen 810 is situated at the upper compartment 808 of the processing device 800 and at least a portion of second bottom basin of the separation screen 810 is position also at the upper compartment 808.

In some embodiments, the top surface of separation screen 810 is perforated and the bottom basin is a sealed surface.

In some embodiments, when separating 810 is elevated, fluids and fecal materials pass through the pores of the top surface of separating screen. When separating 810 is lowered the conveyed fluids and fecal materials are pressed by the bottom basin.

Storage Unit

In some embodiments, fecal materials are removed from fecal processing device 800 to an external storage unit 830. Optionally, storage unit 830 is configured to connect an outlet of device 800 through which fecal materials are removed. In some embodiments, the outlet is valve 820, optionally positioned at the lower section of fecal processing device 800.

General Exemplary Fecal Materials and/or Fluids Movement

In some embodiments, fecal materials and/or fluids flow inside fecal processing device 800 and/or removed from the device subsequent the movement of some mechanical elements in relation to each other within the device.

For example, after fecal materials and/or liquid (e.g., a supportive medium) are introduced into container 828 and placed onto separating screen 810; in the case when the screen is positioned adjacently to blocker 812, the transition of the fecal materials between the upper compartment 808 and the lower compartment 814 is disabled.

Alternatively, subsequent elevation of separating screen and forming a gap between separating screen 810 and blocker 812, the transition of fecal materials and/or fluids is enabled.

In some embodiments, after fecal materials and/or fluids flow from the upper compartment 808 and passes to the lower compartment (e.g., by gravitation), user may lower separating screen 810 (for example by rotating external handle 802) and narrow the gap between the fecal materials and/or liquids which are placed at the lower compartment 814 and the bottom section of the separating screen.

As a gap diminished a positive pressure is form against the fecal materials and/or liquids because of the pressure induced by the bottom surface of separating screen 810, thus fecal materials and/or liquids are pressed towards a filter 816 (optionally positioned at the base of lower compartment 814).

In some embodiments, subsequent transition of fecal materials and/or liquids through filter 816, fecal materials, and/or liquids are collected in a collecting compartment 818 which is positioned under the filter.

In some embodiments, fecal materials and/or liquids may be removed from processing container 828, by draining them through valve 820.

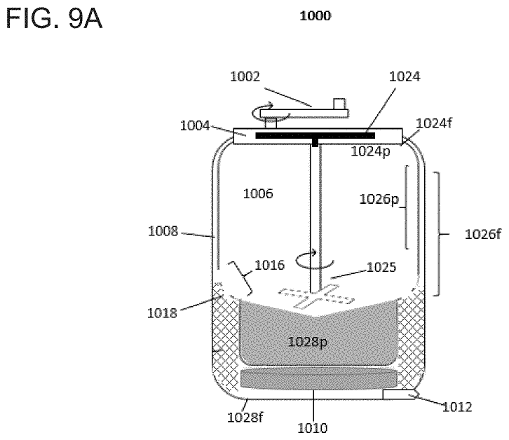

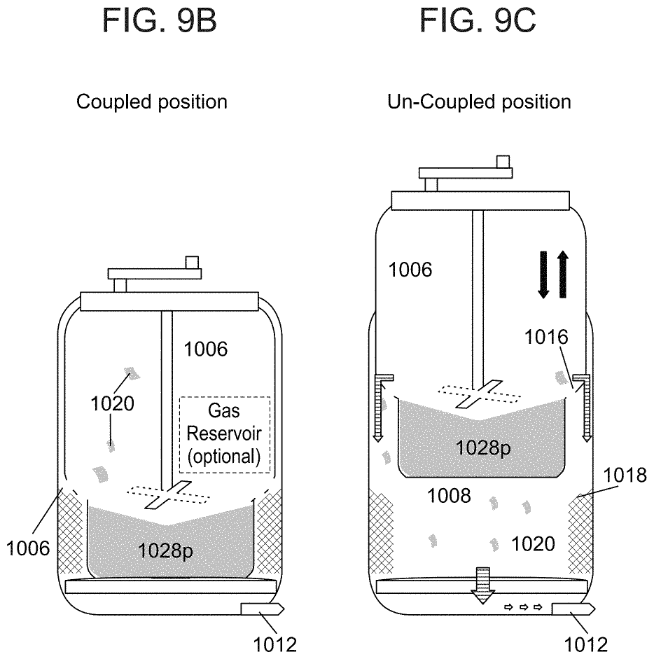

FIG. 9A is a side view of an exemplary fecal processing device 1000, having a processing container positioned inside a filtering container, according to some embodiments of the invention.

In some embodiments, a processing compartment 1006 has a smaller extent than a filtering compartment 1008.

Processing Compartment:

In some embodiments, processing compartment 1006 is defined by an upper opening 1024-p, a side wall 1026-p, and a base 1028-p.

In some embodiments, the processing compartment is cylindrical.

In some embodiments, processing compartment 1006 the side wall, defines an elongated chamber of generally outer circular cross-section. The outer circular cross-section is shaped such that it decreases in cross-sectional size from a large end, to a small end. In some embodiments, the large end is positioned proximal to the upper opening 1024-p. In some embodiments, the small end is positioned proximal to the base 1028-p.

Optionally, both ends are connected to each other by a tilted section, for example, section 1016. In some embodiments, the tilted section is perforated (perforations are represented as a dashed line).

In some embodiments, processing compartment 1006 includes a processing element 1025. Optionally, the processing element 1025 includes a cutting element.

Filtering Compartment:

In some embodiments, filtering compartment 1008 is defined by an upper opening 1024-f, a side wall 1026-f, and a base 1028-f.

In some embodiments, filtering compartment 1008 side wall 1026-f defines an elongated chamber of generally inner circular cross-section. The inner circular cross-section is shaped such that it decreases in cross-sectional size from a large end, to a small end. In some embodiments, the large end is positioned proximal to the upper opening 1024-f. In some embodiments, the small end is positioned proximal to the base.

Optionally, both ends are connected to each other by a tilted section, for example tilted section 1018 (represented by cross-hatching).

According to some embodiments, filtering compartment's 1008 inner wall 1026-f and processing compartment's 1006 outer wall 1026-p are mechanically coupled.

In some embodiments, when processing compartment 1006 is positioned adjacently to the filtering compartment 1008, the tilted section 1018 and the perforated section 1016 are also coupled. When coupled, the apertures positioned at perforated section 1016 are blocked. Optionally, when the tilted section 1018 and the perforated section 1016 are not positioned adjacently, the apertures are open.

In some embodiments, the base 1028-f of filtering compartment 1008 optionally includes a transparent plastic surface.

In some embodiments, filtering compartment 1008 includes a valve 1012. The valve 1012 is optionally positioned at a lower section of the compartment.

In some embodiments, filtering compartment 1008 includes a filter 1110, positioned above the base of the compartment. Optionally, the filtering compartment 1008 includes a valve 1012 through which fecal materials and/or liquids and/or gases are removed from the compartment and/or introduced into the compartment.

In some embodiments, valve 1012 is positioned underneath the filter 1010.

Sealing Lid:

In some embodiments, a sealing lid 1004 is positioned onto the compartments, operable to seal transition of gases and/or fluids and/or solids between the inner sections of both processing compartment 1006 and filtering compartment 1008, and the outer environment.

In some embodiments, sealing lid 1004 comprising an inner compartment that occupies a transmission 1024 positioned therein.

In some embodiments, transmission 1024 transmits rotation of the handle 1002 to the processing element 1025.

Handle:

In some embodiments, rotating handle 1002 is interconnected to sealing lid 1004. Optionally, handle 1002 is connected to the transmission 1024.

In some embodiments, rotation of handle 1002, either clockwise or counterclockwise (presented as a circular arrow positioned on the handle), rotates transmission 1024.

Processing Element:

In some embodiments of the invention, a processing element 1025 is connected to sealing lid 1004. Optionally, processing element 1025 is operable to rotate. (Such rotation is presented as a circular arrow which is marked on the processing element 1025).

In some embodiments, processing element 1025 comprises a shaft having a first end and a second end, the first end is connected to sealing lid 1004.

In some embodiments, the shaft is connected directly to transmission 1024. Processing element 1025 may include a cutting element, for example, a cutting blade.

Filter:

In some embodiments of the invention, exemplary filter 1010 includes pores having a characterizing extent of between 0.1 to 2 mm is provided.

Optionally, a maximal extent of the pores is between 0.8 mm to 1.8 mm, for example a range between 0.9 to 1.8 mm, 1-2 mm, 1.2-1.8 mm or any intermediate, smaller or larger value, or range of values.

Elements Movement:

Referring now to FIGS. 9B and 9C. These two figures show an exemplary side view of a fecal processing device 1000, having processing container 1006 positioned inside filtering container 1008 in a coupled position (FIG. 9B) and in an un-coupled position (FIG. 9C), according to some embodiments of the invention

In some embodiments of the invention, exemplary processing compartment 1006 is operable to move inside filtering compartment 1008. Optionally, the movement resembles a piston (processing compartment 1006) operable to move inside a cylinder (filtering compartment 1008). Such movement is exemplified by two opposite arrows presented in FIG. 9C.

In some embodiments, processing compartment 1006 and filtering compartment 1008 walls are sized and shaped to allow a continuous contact between the walls of each compartment. In some embodiments, when processing compartment 1006 is placed inside filtering compartment 1008 and when perforated section 1016 and tilted section 1018 touch each other, the pores of perforated section 1016 are blocked (FIG. 9B). When processing compartment 1006 is elevated, a gap between perforated section 1016 and tilted section 1018 is formed (FIG. 9C). In such case, the pores of perforated section 1016 are open.

Fecal Materials and/or Fluids Transition:

In some embodiments, the pores of perforated section 1016 may have an extent in a range between 2 mm to 8 mm.

Optionally, a maximal extent of the pores is between 3 mm to 8 mm, for example in a range between 4 to 8 mm, 5-8 mm, 6-8 mm or any intermediate, smaller or larger value, or range of values.

When a gap between the tilted section 1018 and perforated section 1016 is formed, fecal materials and/or liquids passes from processing compartment 1006 downwards to the filtering compartment 1008 through the pores of the perforated section 1016.

In some embodiments, only fecal materials and/or solid parts having an extent which is smaller than the extent of the pores, manage to pass through the pores to the filtering compartment 1008 and fecal materials and/or solid parts having a larger extent remain in the processing compartment 1006. A potential advantage of preventing the transition of large pieces (either large fecal particles and/or large undigested food particles) is avoiding blockage of the filter 1010.

Fecal Materials Compression:

In some embodiments, processing compartment 1006 includes a base 1028p. Optionally, the base has an impermeable rigid surface.

In some embodiments, after fecal materials and/or fluids flow from the processing compartment 1006 and pass to the filtering compartment 1008 (e.g., by gravitation), user may lower processing compartment 1006. When the processing compartment is lowered, fecal materials and/or fluids are pressed by the impermeable rigid base 1028p towards filter 1010.

Fecal materials removal from fecal processing device:

In some embodiments, after the transition of fecal materials and/or liquids through filter 1010, fecal materials, and/or liquids are removed from processing device 1000, by draining them through tap and/or valve 1012.

Gas Insertion:

In some embodiments of the invention, gas is introduced into processing device 1000. Optionally, gas is introduced to the processing compartment 1006. Optionally or alternatively, gas is introduced to the filtering compartment 1008.

In some embodiments, gas is introduced through a valve. Optionally or alternatively, gas is injected by a syringe (not shown).

Optionally or alternatively, processing device 1000 includes a gas reservoir. Optionally, a gas reservoir is positioned inside the container (processing and/or filtering container).

In some embodiments, the gas is released from a bag, upon activation of a processing element 1025 which may have a cutting element. Optionally, such cutting element is operable to tear the bag and release its gas content.

Optionally or alternatively, the gas reservoir is positioned inside the walls of processing compartment 1006 and/or inside the walls of filtering compartment 1008.

Optionally, the reservoir is being separated from the inner environment of either the processing compartment 1006 and/or filtering compartment 1008 by a separating membrane (not shown). Optionally, activation of processing element 1025 may tear the membrane to allow releasing of the gas.

### Extracting/Transplanting Processed Fecal

As disclosed elsewhere herein, fecal material produced within the fecal processing device can be stored in a store unit and transplanted after being stored.

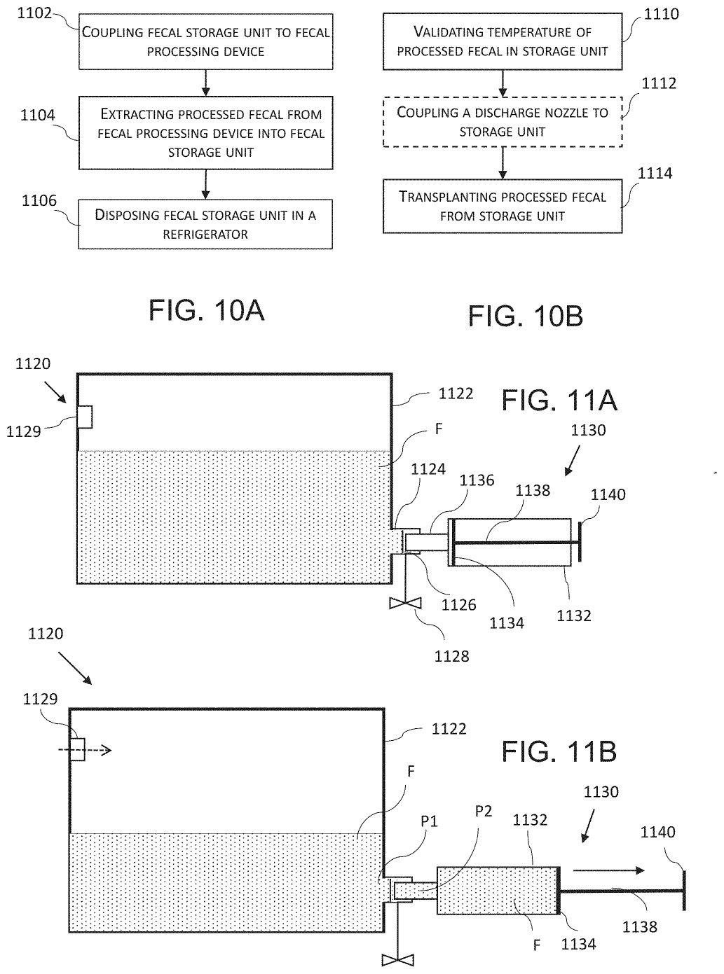

Turning to FIG. 10A, which is a simplified flowchart of a process of storing processed fecal, according to some embodiments of the invention.

According to some embodiments, storing of fecal processed by a fecal processing device includes coupling 1102 the storage unit to the fecal processing device, extracting 1104 the processes fecal into the storage unit, and disposing 1106 the storage unit in a temperature controlled environment.

According to some embodiments, the process of storing fecal is performed by a patient. In some embodiments, the process of storing fecal is performed by a professional.

According to some embodiments, coupling 1102 is by connecting a port at the storage unit to an outlet at the processing device. In some embodiments, coupling 1102 is by screwing a port at the storage unit to an outlet at the processing device. In some embodiments, coupling 1102 is by pressing a port at the storage unit into/over an outlet at the processing device. In some embodiments, coupling 1102 is by an adaptor interconnecting the processing device and the storage unit. According to some embodiments, coupling 1102 is without leaking processed fecal outside the processing device and the storage unit.

According to some embodiments, extracting 1104 is by generating positive pressure differential between the fecal processing device and the storage unit. In some embodiments, extracting 1104 includes applying suction at an outlet of the fecal processing device by the storage unit. In some embodiments, a positive pressure is generated inside the fecal processing device prior and/or during extracting 1104 to reduce the suction force required to be applied for conveying proceeded fecal into the storage unit. In some embodiments, the positive pressure inside the fecal processing device is higher than the pressure within the storage unit, so that conveying proceeded fecal into the storage unit does not require suction to be applied.

According to some embodiments, disposing 1106 is in a temperature controlled environment. In some embodiments, the temperature controlled environment is a refrigerator. In some embodiments, the temperature controlled environment is a heat isolated compartment. In some embodiments, disposing 1106 is in a compartment fitted to hold and isolate the storage units of other items in the refrigerating system.

According to some embodiments, there are optional steps in the process of storing processed fecal. In some embodiments, storing include detaching some components of the storage unit for fitting within the temperature controlled environment. In some embodiments, detaching includes removing a handle used to apply suction during extraction.

According to some embodiments, extracting 1104 is followed or includes mixing the processed fecal within the fecal storage unit. In some embodiments, the fecal storage unit is configured to mix the processed fecal within the fecal storage unit during extracting 1104. In some embodiments, the fecal storage unit is configured to mix the processed fecal within the fecal storage unit following extracting 1104.

According to some embodiments, a step of filling the storage unit with a fluid precedes coupling 1102. In some embodiments, the fluid is a preservation material. In some embodiments, the fluid includes medication. In some embodiments, fluid is for improving releasing fecal out of the storage unit during transplantation.

According to some embodiments, the steps of coupling 1102 to disposing 1106 are repeated to prepare required amount of processed fecal.

Turning to FIG. 10B, which shows a flowchart of a method of transplanting processed fecal stored in a fecal storage unit, according to some embodiments of the invention.

According to some embodiments, transplanting a stored processes fecal to a consuming user is by injecting the stored processes fecal. In some embodiments, transplanting is a rectal transplantation. According to some embodiments, transplanting is of a processed fecal of the source user who produced the fecal. In some embodiments, transplanting is part of a medical treatment of a consuming user. According to some embodiments, transplanting is to a consuming user who is not the source who produced the fecal. In some embodiments, transplanting is part of a wellbeing procedure, which is not part of a medical treatment.

According to some embodiments, the transplanting process includes the following steps:

Validating 1110 temperature of processed fecal in fecal storage unit. According to some embodiments, validating 1110 is by reading a temperature indication provided at the storage unit.

Coupling 1112 a discharge nozzle to storage unit. According to some embodiments, coupling 1112 discharge nozzle improves the performance of transplanting. In some embodiments, coupling 1112 discharge nozzle improves access to the rectum. In some embodiments, coupling 1112 discharge nozzle adds comfort to the patient. In some embodiments, coupling 1112 is to reduce leakage of fecal during transplanting. According to some embodiments, coupling 1112 is optional. In some embodiments, the storage unit includes a tip, which provides an alternative to one or more of the functions of the nozzle and reduces the need of coupling 1112 a discharge nozzle.

Optionally, coupling 1112 is preceded by mixing fecal within the storage unit. Optionally, coupling 1112 is preceded by heating fecal within the storage unit.

Transplanting 1114 processed fecal from the storage unit. In some embodiments, transplanting 1114 is after coupling the storage unit to the body of the patient. In some embodiments, transplanting 1114 is after taking medication by the consuming user.

According to some embodiments, coupling 1112 includes interconnecting an adaptor between the discharge nozzle and the storage unit. In some embodiments, the adaptor is long enough to enable holding the storage unit by the patient while a discharge nozzle is couple to the rectum of the patient during transplanting 1114.

According to some embodiments, the steps of validating 1110 to transplanting 1114 are repeated according to the amount of fecal required to be received by the receiving user.

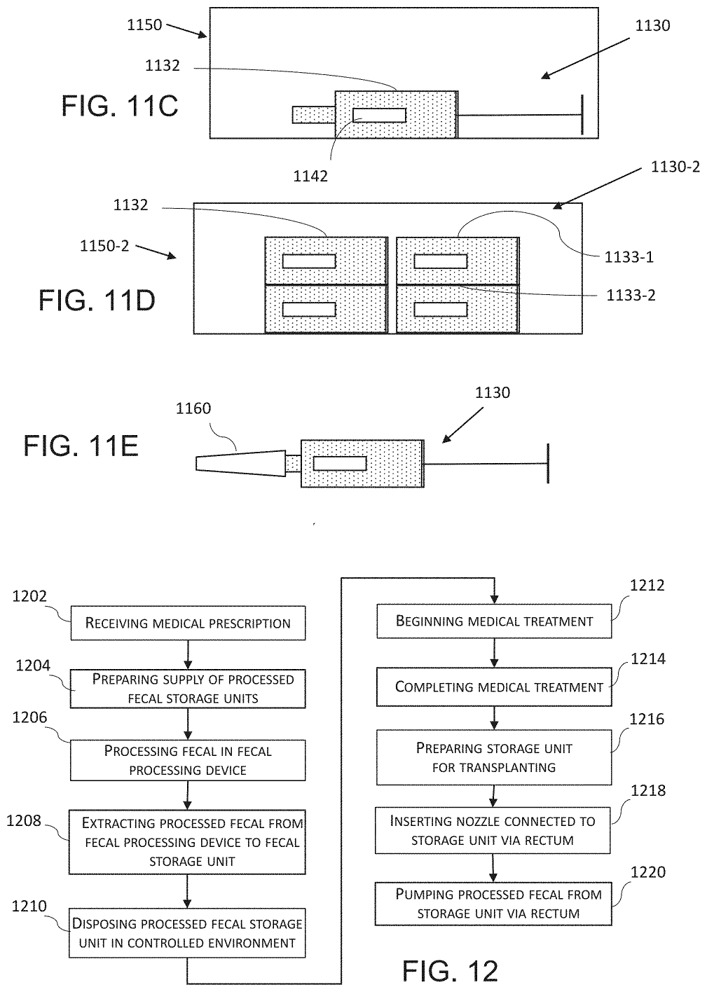

Turning to FIGS. 11A to 11E, which show schematic illustrations of a fecal storage unit, according to some embodiments of the invention.

According to some embodiments, processed fecal material F is transferred from fecal processing device 1120 to a storage unit 1130.

According to some embodiments, as shown for example in FIGS. 11A and 11B, fecal processing device 1120 includes a container 1122 and one or more outlets 1124 disposed at one or more walls of container 1122. In some embodiments, container 1122 includes a compartment for receiving processed fecal material. In some embodiments, outlet ports 1124 are disposed at one or more walls of the compartment. In some embodiments, one or more outlets 1124 protrude outside container 1122. In some embodiments, one or more outlets 1124 are formed at container 1122 without protruding outside container 1122. According to some embodiments, one or more outlets 1124 are configured to prevent leakage of processed fecal. In some embodiments, one or more of outlet ports 1124 include a valve 1126 to prevent leakage.

According to some embodiments, outlet ports 1124 include one way valve to allow conveying processed fecal outside of container 1122. In some embodiments, valve 1126 self-opens at a positive pressure differential (P1>P2 directed outside processing device 1120). In some embodiments, valve 1126 opens at a pressure differential (P1−P2). In some embodiments, device 1120 includes a manual valve 1128 to control the conveying of processed fecal via port 1124. In some embodiments, processing device 1120 includes a pressure valve 1129 for allowing air into container 1122 to maintain a positive pressure inside container 1122. In some embodiments, pressure valve 1129 is a one-way valve.

A potential advantage of processing fecal and reducing particles size is reducing the pressure differential required to be applied in order to extract process fecal F through small outlet ports 1124. Another potential advantage of processing fecal and reducing particles size is reducing the size of outlet ports 1124 and/or the size of port 1136, since particles are small enough to flow through outlet ports 1124 and/or the size of port 1136 without clogging the ports.

According to some embodiments, the maximal width of the opening of outlet port 1124 is between 0.5 mm and 8 mm. In some embodiments, the maximal width of the opening of outlet port 1124 is between 1 to 6 mm. In some embodiments, the maximal width of the opening of outlet port 1124 is between 2 to 5 mm. According to some embodiments, the maximal width of the opening of port 1136 is between 0.5 mm and 8 mm. In some embodiments, the maximal width of the opening of port 1136 is between 1 to 6 mm. In some embodiments, the maximal width of the opening of port 1136 is between 2 to 5 mm.

According to some embodiments, storage unit 1130 has a hollow body 1132 and a suction mechanism 1134. According to some embodiments, the storage unit 1130 includes a manual pump to apply a negative pressure at outlet 1124 (from outside direction of outlet 1124). In some embodiments, as shown for example in FIG. 11B, suction mechanism 1134 is functionally similar to syringe, having a suction port 1136, a plunger 1138, and a handle 1140. In some embodiments, suction mechanism 1134 is squeezable. In some embodiments, suction mechanism 1134 includes a squeezable bulb to apply suction and/or pressure at port 1136 (e.g. as item 650 shown in FIG. 6B).

According to some embodiments, suction port 1136 has a protruding tip. In some embodiments, port 1136 is formed at the surface of body 1132 (e.g. like a syringe without a protruding tip). In some embodiments, port 1136 is sized to fit tightly inside outlet 1124. In some embodiments, port 1136 is adapted to fit tightly outside over outlet 1124. In some embodiments, port 1136 includes a mechanical indicator to inform the user about securing port 1136 to outlet 1124. In some embodiments, port 1136 snap fit to outlet 1124. In some embodiments, securing port 1136 to outlet 1124 provides a “click” sound. According to some embodiments, outlet 1124 is sized to fit tightly to port 1136. In some embodiments, outlet 1124 is adapted to fit tightly outside over port 1136. In some embodiments, outlet 1124 includes a mechanical indicator to inform the user about securing outlet 1124 to port 1136. In some embodiments, outlet 1124 snap fits to port 1136. In some embodiments, securing outlet 1124 to port 1136 provides a “click” sound.

According to some embodiments, storage unit 1130 is configured to mix extracted processed fecal F. In some embodiments, storage unit 1130 is configured to mix the processed fecal F during extracting. In some embodiments, suction mechanism 1134 has one or more rotatable portions, to mix processed fecal F disposed within storage unit 1130. In some embodiments, plunger 1138 is rotatable within body 1132. In some embodiments, body 1132 is shaped to rotate plunger 1138 during an axial motion of plunger 1138 inside body 1132.

According to some embodiments, as shown for example in FIG. 11C, storage unit 1130 includes an indicator 1142 to indicate a state of the processes fecal. In some embodiments, indicator 1142 is a temperature gauge.

According to some embodiments, storage unit 1130 includes a sealing bag. In some embodiments, there is provided a processed fecal storage kit, having one or more storage units 1130 and one or more sealing bags.

According to some embodiments, storage unit 1130 body 1132 includes a compartment for storing processed fecal F. In some embodiments, the maximal volume of the compartment is between 50 and 300 cc. In some embodiments, the maximal volume of the compartment is between 100 and 250 cc. In some embodiments, the maximal volume of the compartment is between 150 and 200 cc.

According to some embodiments, as shown for example in FIGS. 11C and 11D, storage unit 1130 is shaped and sized for a vertical piling within an insulated compartment 1150/1150-2 (e.g. a refrigerator, a cooling box). In some embodiments, body 1132 is shaped to allow a stable vertical accumulation of storage units 1130 one on top another within insulated compartment 1150. In some embodiments, body 1132 has a top flat surface 1133-1 and a bottom flat surface 1133-2. In some embodiments, top flat surface 1133-1 and a bottom flat surface 1133-2 are parallel.

According to some embodiments, as shown in FIG. 11D, storage unit 1130 has one or more detachable components to reduce the volume of storage unit 1130 occupies within controlled environment 1150-2. In some embodiments, plunger 1138 and/or handle 1140 is detachable.

According to some embodiments, as shown for example in FIG. 11E, the processed fecal storage kit includes a discharge nozzle 1160 configured for coupling to storage unit 1130. A potential advantage of coupling a discharge nozzle 1160 is improving the performance of transplanting. Another potential advantage is increasing transplanting comfort to the patient. Another potential advantage is reducing leakage of fecal during transplanting. Another potential advantage is reducing the size of storage unit 1130, to have a reduced length when an extended port 1136 or a discharge nozzle 1160 are not required. In some embodiments, port 1136 of storage unit 1130 includes a tip, which provides an alternative to one or more of the functions of discharge nozzle 1160 and reduces the need of coupling a discharge nozzle 1160 to unit 1130.

According to some embodiments, the processed fecal storage kit includes a fluid to insert inside the storage unit 1130. In some embodiments, the fluid is a medication. In some embodiments, the fluid is a preservation material.

Turning to FIG. 12, which is a simplified flowchart of a process of storing and transplanting processed fecal, according to some embodiments of the invention.

According to some embodiments, transplanting a processed fecal is part of a medical treatment. In some embodiments, transplanting is part of a treatment, which is not medical and does not require prescription. The following are some steps of a transplanting process:

Receiving 1202 medical prescription. In some embodiments, prescription is of antibiotics. In some embodiments, prescription includes a treatment protocol.

Preparing 1204 supply of fecal storage units. In some embodiments, preparing 1204 is according to a treatment protocol. The storage units can be any one of the storage units described elsewhere herein. In some embodiments, storage units are storage units adapted to be sorted in a home refrigerator. In some embodiments, storage units are kits of storage units as described elsewhere herein.

Processing 1206 fecal in fecal processing device. In some embodiments, processing is according to processing methods described elsewhere herein (e.g. in FIGS. 3 and 4). In some embodiments, processing 1206 is by a fecal processing device described elsewhere herein. In some embodiments, processing 1206 is by a user who is a patient. In some embodiments, processing 1206 is by a user who is a source user.

Extracting 1208 processed fecal from fecal processing device to fecal storage unit. In some embodiments, extracting 1208 is as described elsewhere herein (e.g. in FIG. 10A and FIG. 4). In some embodiments, extracting 1208 is by a user who is a patient. In some embodiments, extracting 1208 is into 2 to 10 storage units. In some embodiments, extracting 1208 is into 3 to 8 storage units. In some embodiments, extracting 1208 is into 4 to 6 storage units. In some embodiments, the processing device is disposable and being disposed after extracting 1208.

Disposing 1210 processed fecal storage unit in controlled environment. Alternatively, disposing 1210 is optional, for example when treating a receiving user who is not the source user.

Beginning 1212 medical treatment. In some embodiments, medical treatment requires taking antibiotic medication. In some embodiments, the medication treatment is of the source user. In some embodiments, the medication treatment is of a destination user, who is not the source user.

Completing 1214 medical treatment. In some embodiments, completing 1214 medical treatment includes terminating medication treatment. Alternatively, beginning 1212 and completing 1214 treatment are optional, for example when transplanting the processed fecal material to a receiving user who is not the source user.

Preparing 1216 one or more of the stored storage units for fecal transplanting. In some embodiments, preparing 1216 includes attaching one or more components to the storage units. In some embodiments, preparing 1216 includes heating the storage unit. In some embodiments, preparing 1216 includes mixing content within the storage unit. In some embodiments, preparing 1216 includes adding fluid within storage unit.

Inserting 1218 nozzle connected to storage unit via rectum. In some embodiments, nozzle is part of a kit provided with the storage unit and/or processing device. In some embodiments, inserting 1218 is preceded by coupling nozzle to the storage device.

Pumping 1220 processed fecal from storage unit via rectum. In some embodiments, pumping 1220 is by the user who receives the processed fecal. In some embodiments, pumping 1220 is manual by the user proving pumping energy to the storage unit. In some embodiments, pumping 1220 is by activating a pump. In some embodiments, pumping 1220 is by an operator, who is not the receiving user.

According to some embodiments, the steps of preparing 1216 to pumping 1220 are repeated according to the amount of fecal required to be received by the receiving user. In some embodiments, the steps of preparing 1204 to disposing 1210 are repeated to prepare required amount of processed fecal according to a treatment protocol. One or more of the steps can be optional (form example, receiving 1202, preparing 1204, and inserting 1218). In some embodiments, one or more of the steps can be performed in a different order, for example preparing 1204 can be performed prior to processing 1206.

According to some embodiments, the treatment protocol includes administrating 3 to 5 storage units of fecal material by 24 hours of the medical treatment (e.g. after completing 1212 medical treatment). In some embodiments, the treatment protocol includes a daily administration of 2 to 5 storage units of fecal material after the medical treatment (e.g. after completing 1212 medical treatment).

### General

It is expected that during the life of a patent maturing from this application many relevant fecal processing devices will be developed; the scope of the term fecal processing is intended to include all such new technologies a priori.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, stages and/or parts, but only if the additional ingredients, stages and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers there between.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority documents of this application are hereby incorporated herein by reference in their entirety.

1. A method of producing an implant for fecal transplant (FT), comprising:
introducing fecal materials into a container comprising a first compartment and a second compartment, and a separating screen separating said compartments;
processing in non-ambient conditions said fecal materials to a smaller size in said container;
conveying said fecal materials through a plurality of apertures located in said separating screen to said second compartment.

2. The method of claim 1, wherein said non-ambient conditions comprise an atmosphere having at most 18% of O₂.

3-4. (canceled)

5. The method of claim 1, comprising sealing said container with a sealing lid after said introducing, and processing said fecal materials in said sealed container.

6-7. (canceled)

8. The method of claim 32, wherein said processing comprises moving said separating screen in relation to a blocker, to allow transition of said fecal materials from the upper compartment to the lower compartment.

9. The method of claim 8, wherein said method comprises:
a) sealing said container with a sealing lid, and connecting a handle to said sealing lid;
b) after a), rotating said handle to rotate a processing element; and
c) after b), rotating said handle to move said separating screen in relation to said blocker.

10-12. (canceled)

13. The method of claim 8, comprising directing said fecal materials along an inner perimeter of said blocker, said inner perimeter having a funneled geometry to allow conveyance of said fecal materials toward a filter, wherein said blocker is positioned below said separating screen.

14-17. (canceled)

18. Apparatus for producing implant for fecal transplant (FT), comprising:
a container sized to receive a sample of fecal materials; and
a size reducer positioned in said container and operable to reduce the size of said fecal materials;
wherein, said container comprises a separating screen having a plurality of apertures, which divides said container into a first upper compartment comprising said size reducer and to a second lower compartment; and
wherein said plurality of apertures in said separating screen allow transition of liquids and fecal materials between said first upper compartment and said second lower compartment.

19. The apparatus of claim 18, comprising a sealing lid, said sealing lid is sized and shaped to prevent transition of gases and liquids between an inner section of said container and outer environment.

20. The apparatus of claim 18, wherein said size reducer comprises at least one rotating cutting blade.

21. The apparatus of claim 33, wherein said container comprises a collecting compartment, said collecting compartment is positioned under said lower compartment and separated from said lower compartment by a filter.

22. The apparatus of claim 33, wherein said plurality of apertures have a maximal extent of at most 8 mm, and are selectively blocked by said blocker.

23. The apparatus of claim 18, wherein said container is shaped and sized for insertion into a toilet bowl.

24. The apparatus of claim 19, wherein said sealing lid comprising a handle attached to said lid, and wherein said lid comprises:
a first top surface;
a second bottom surface; and
a transmission positioned between the first top surface and the second bottom surface, and the transmission transfers a rotation of said handle to said size reducer.

25. The apparatus of claim 21, wherein said blocker has an inner perimeter having a conical shape, configured to direct said liquids and fecal materials gravitationally toward said filter.

26-27. (canceled)

28. The apparatus of claim 22, wherein said separating screen is coupled to a shaft, wherein said shaft is configured to move said separating screen in relation to said blocker.

29. The apparatus of claim 24, wherein said size reducer is interconnected to said transmission, and said transmission is configured to rotate said size reducer.

30. (canceled)

31. A fecal transplantation kit, comprising:
a fecal processing apparatus according to claim 34; and
one or more storage units, comprising a port sized and shaped to be secured to said one or more outlets without a leakage of said fecal material.

32. The method of claim 1, wherein said first compartment is an upper compartment, and said second compartment is a lower compartment, and wherein said conveying comprises conveying said fecal materials through said plurality of apertures downward to said lower compartment.

33. The apparatus of claim 18, wherein said separating screen is positioned onto a blocker, and wherein said separating screen is movable in relation to said blocker to allow said transition of liquids and said fecal material through said plurality of apertures between said first upper compartment and said second lower compartment.

34. The apparatus of claim 18, comprising:
one or more outlets at one or more walls of said second lower compartment to extract said fecal materials out of said container.
